SEQUENCE LISTING (16 sequences)

SEQ ID NO: 1 - DNA, 20 bp, Artificial Sequence
  primer
gagtcaacggatttggtcgt

SEQ ID NO: 2 - DNA, 20 bp, Artificial Sequence
  primer
ttgattttggagggatctcg

SEQ ID NO: 3 - DNA, 18 bp, Artificial Sequence
  primer
cgcacggaactcctgacc

SEQ ID NO: 4 - DNA, 18 bp, Artificial Sequence
  primer
gccaccaccaccatctcg

SEQ ID NO: 5 - DNA, 20 bp, Artificial Sequence
  primer
atggtgtgcgagtggtcaat

SEQ ID NO: 6 - DNA, 20 bp, Artificial Sequence
  primer
accacagttccatgcaccaa

SEQ ID NO: 7 - DNA, 20 bp, Artificial Sequence
  primer
gaggtcctgagcgagttcga

SEQ ID NO: 8 - DNA, 20 bp, Artificial Sequence
  primer
tctctgtttcaggccgaaca

SEQ ID NO: 9 - DNA, 16 bp, Artificial Sequence
  primer
gcccgcagcctagcaa

SEQ ID NO: 10 - DNA, 22 bp, Artificial Sequence
  primer
cggtaaaagatcccgcatgtag

SEQ ID NO: 11 - DNA, 20 bp, Artificial Sequence
  primer
ctggtggacctcatcgaaca

SEQ ID NO: 12 - DNA, 23 bp, Artificial Sequence
  primer
cgtctcgttcagatccttttcct

SEQ ID NO: 13 - DNA, 19 bp, Artificial Sequence
  primer
gcagctatcaaccagattc

SEQ ID NO: 14 - DNA, 20 bp, Artificial Sequence
  primer
gatgtaggcagctgtcattc

SEQ ID NO: 15 - DNA, 20 bp, Artificial Sequence
  primer
ccagcaagcacaaaatttca

SEQ ID NO: 16 - DNA, 21 bp, Artificial Sequence
  primer
tgcactggatctgtttcttca

(19) United States
(12) Patent Application Publication
(10) Pub. No.: US 2024/0240143 A1
(43) Pub. Date: Jul. 18, 2024

(54) BIOENGINEERED DERMAL PAPILLA AND HAIR FOLLICLES AND RELATED PRODUCTS, METHODS AND APPLICATIONS

(71) Applicant: The University of Hong Kong, Hong Kong (CN)

(72) Inventors: Pui Barbara Chan, Hong Kong (CN); Wan Jing Ou, Hong Kong (CN)

(21) Appl. No.: 18/564,498

(22) PCT Filed: May 27, 2022
(86) PCT No.: PCT/CN2022/095588

§ 371 Date: Nov. 27, 2023

Related U.S. Application Data
(60) Provisional application No. 63/194,063, filed on May 27, 2021.

(51) Int. Cl. C12N 5/071; A61K 35/36

(52) U.S. Cl. CPC C12N 5/0629; A61K 35/36; C12N 2503/02; C12N 2513/00; C12N 2533/54; C12N 2533/90

(57) ABSTRACT

Compositions and methods involving microspheres composed of mesenchymal cells such as dermal papilla cells (DPCs) and extracellular matrix, epithelial cell such as keratinocyte are disclosed. It has been discovered that by balancing the proportion of DPCs and extracellular matrix in the DPC-matrix mixture, and by incubating a particular range of small volume of the DPCmatrix mixture, DPC-matrix microspheres can be formed that have useful properties. Most significantly, the resulting DPC-matrix microspheres are particularly suited to be used to produce bioengineered hair follicles that have features of native hair follicles.

This international patent application claims the benefit of U.S. Provisional Patent Application No. 63/194,063 filed on May 27, 2021, the entire content of which is incorporated by reference for all purpose.

### FIELD OF THE INVENTION

The disclosed invention is generally in the field of bioengineered tissue and specifically in the area of bioengineered hair follicle tissue.

### BACKGROUND OF THE INVENTION

Hair follicle (HF) is a complex “mini-organ” consisting of multiple types of cells, including epithelial, mesenchymal, and neural crest cells. One population of mesenchymal cells in the HFs, dermal papilla (DP) cells, is regarded as essential for inducing the epithelial cells during HF formation and postnatal hair growth (Driskell et al., 2011). Appropriate communication and interactions between the epithelial and mesenchymal originated cells spatially and temporally result in the generation, maintenance, and renewal of hair during development, growth, and wound repair. During the early stage of HF development, DP is formed as a mesenchymal condensate, which shrinks to a small ball of cells, dropping to the base of the hair follicle and remain embedded throughout the hair growth period. DP continues to signal the epithelial compartment by expressing signaling molecules involving Wnt, FGF, noggin, and SHH, promoting HF formation, regeneration, and subsequent hair growth (Ohyama et al., 2010; Reddy et al., 2001).

Although there is no new hair follicle formation after birth, the lower part of the hair follicle keeps a cyclic growth to generate new hair shafts throughout the lifetime, from regeneration (anagen), regression (catagen) to quiescence (telogen). However, progressive miniaturization of HFs caused by internal or external triggers will lead to a shortened anagen period and a decreased number of hairs, and eventually develops into hair loss (Premanand & Reena Rajkumari, 2018).

Hair not only has useful biological functions like protection from the harmful elements and dispersion of sweat-gland products but also has psychosocial importance in our society. Although hair loss itself is not life-threatening, affected individuals always experience great psycho-emotional stress and may have psychological and/or psychiatric problems such as anxiety, distress, and depression, which significantly undermines their quality of life (Aghaei et al., 2014; Cartwright et al., 2009; Gokalp, 2017; Hunt & McHale, 2005; Williamson et al., 2001).

Numerous treatments for hair loss including medications, plant extracts, and phototherapy have been developed, but most of the current alopecia treatments are far from satisfactory, either entailing considerable side effects or having limited and temporary effects, mostly targeting patients with mild alopecia symptoms (Sadick et al., 2017). The surgical treatment uses small pieces of hair follicles harvested from a safe scalp area for autologous hair transplantation. However, the success of transplantation relies on sufficient donor supply, where the limited availability of donor HFs is one of the bottlenecks, not to mention and the painful harvesting procedure, the labor-intensiveness, and high cost of the procedure.

The limitation of existing treatments has driven the search for better therapeutic alternatives to address this unmet medical need, among which stem cell-based tissue engineering is emerging as a promising approach. Although de novo hair regeneration in murine skin has demonstrated success using murine follicular dermal papilla cells combined with epithelial cells, reconstitution of human hair follicles has not yet been successful (Castro & Logarinho, 2020). Attempts to generate bioengineered human hair follicles that recapitulate key hair-specific markers and hair induce potential have been difficult due to the complicated three-dimensional organization and multiple signaling interplays. Challenges to establishing a physiologically relevant engineered hair follicle include the maintenance of human hair follicle cell phenotypes, the development of defined culture conditions incorporating different niche factors, and proper structural design that enables effective epithelial-mesenchymal interactions and mimics the three-dimensional configurations of human hair follicles.

It is therefore an object of the present invention provide methods of preparing bioengineered hair follicles.

It is a further object of the present invention to provide bioengineered hair follicles.

It is a further object of the present invention to provide methods of using bioengineered hair follicles in vitro.

It is a further object of the present invention to provide methods of using bioengineered hair follicles in vivo.

It is a further object of the present invention to provide methods of using bioengineered hair follicles to identify drugs and therapies for treatment of native hair follicles and hair loss.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### BRIEF SUMMARY OF THE INVENTION

Disclosed are compositions and methods involving microspheres composed of mesenchymal cells and extracellular matrix, keratinocyte-containing forms of such microspheres, and bioengineered hair follicles produced from such microspheres. It has been discovered that by balancing the proportion of mesenchymal cells and extracellular matrix in the mesenchymal cell-matrix mixture, and by incubating a particular range of small volume of the mesenchymal cell-matrix mixture, mesenchymal cell-matrix microspheres can be formed that have useful properties. Most significantly, the resulting mesenchymal cell-matrix microspheres are particularly suited to be used to produce bioengineered hair follicles that have features of native hair follicles.

Disclosed are methods of producing a bioengineered hair follicle, the method comprising
forming a microsphere comprising mesenchymal cells and extracellular matrix by dispensing a droplet of a suspension of the mesenchymal cells and extracellular matrix into a vessel and incubating the droplet, thereby forming a mesenchymal cell-matrix microsphere;
culturing the mesenchymal cell-matrix microsphere in the vessel in the presence of supplementary factors;
dispensing to the vessel a droplet of a suspension of epithelial cells in close proximity to the mesenchymal cell-matrix microsphere to form a mesenchymal microsphere-epithelial cell mixture, and culturing the mesenchymal microsphere-epithelial cell mixture; and
changing the medium in the vessel to epidermalization medium and culturing, thereby producing a bioengineered hair follicle.

In some forms, and more specifically, the method can comprise forming a microsphere comprising mesenchymal cells and extracellular matrix by dispensing a droplet of a suspension of the mesenchymal cells and extracellular matrix into a vessel, and incubating the droplet at a temperature of from 25° C. to 39ºC, from 35° C. to 39° C., or preferably 37° C., in a humidified atmosphere optionally with from 3.5% to 6% CO₂ for from 1 hour to 100 hours, from 5 hours to 50 hours, or preferably from 8 hours to 30 hours, in a culture vessel, thereby forming a mesenchymal cell-matrix microsphere;
culturing the mesenchymal cell-matrix microsphere in the vessel in the presence of supplementary factors;
dispensing to the vessel a droplet of a suspension of epithelial cells in close proximity to the mesenchymal cell-matrix microsphere to form a mesenchymal microsphere-epithelial cell mixture, and culturing the mesenchymal microsphere-epithelial cell mixture; and changing the medium in the vessel to epidermalization medium and culturing at from 35° C. to 39ºC in a humidified atmosphere optionally with from 3.5% to 6% CO₂ for from 1 to 20 days, or preferably from 3 to 10 days, thereby producing a bioengineered hair follicle.

In some forms, the droplet of the suspension of the mesenchymal cells and extracellular matrix has a volume ranging from 0.5 to 10.0 μL, from 1.0 to 5.0 μL, or preferably from 2.0 to 3.0 μL. In some forms, the suspension of the mesenchymal cells and extracellular matrix comprises the mesenchymal cells at a density of from 1×10⁴ to 1×10⁷ cells/ml, or preferably 1×10⁵ to 1×10⁶ cells/ml, and extracellular matrix at a concentration of from 0.01 mg/ml to 2.0 mg/ml, or preferably from 0.05 mg/ml to 0.5 mg/ml.

In some forms, the mesenchymal cells are human dermal papilla cells (DPCs), human mesenchymal stem cells, human fibroblasts, or a combination thereof. In some forms, the extracellular matrix comprises collagen, fibronectin, fibrinogen, laminin, glycosaminoglycans, vitronectin, or a combination thereof. In some forms, the extracellular matrix comprises or substantially consists of collagen.

In some forms, the culture vessel is comprised in a culture platform, wherein the culture platform is a 384 well culture plate, a custom-made 88 well microwell, or a PDMS-based microwell. In some forms, the supplementary factors comprise FGF, HGFs, Wnt, BMP, PDGF, or a combination thereof.

In some forms, the mesenchymal cell-matrix microsphere is cultured in the vessel in the presence of supplementary factors at from 25° C. to 39° C., preferably 37° C. in a humidified atmosphere with from 3.5% to 6% CO₂ for from 1 to 100 hours, preferably from 12 hours to 30 hours.

In some forms, the droplet of the suspension of the epithelial cells has a volume ranging from 0.5 to 10.0 μL, 1.0 to 5.0 μL, or preferably from 2.0 to 3.0 μL, and preferably the suspension contains the epithelial cells at a density of from 1×10⁴ to 1×10⁷ cells/ml, or preferably 1×10⁵ to 1×10⁶ cells/ml. In some forms, the mesenchymal microsphere-epithelial cell mixture is cultured at from 35° C. to 39ºC in a humidified atmosphere with from 3.5% to 6% CO₂ for from 1 hours to 100 hours, 5 hours to 50 hours, or preferably 18 hours to 30 hours. In some forms, the epithelial cells are human epidermal keratinocytes, human hair follicle keratinocytes, human epidermal progenitor cells, human iPSC derived epithelial cells, or a combination thereof.

In some forms, all of the incubations and culturings are performed at 37° C. in a humidified atmosphere with 5% CO₂. In some forms, the droplets of mesenchymal cells and extracellular matrix are incubated overnight, wherein the mesenchymal cell-matrix microsphere is cultured overnight, and wherein the mesenchymal cell microsphere-epithelial cell mixture is cultured overnight. In some forms, the mesenchymal cell microsphere-epithelial cell mixture is cultured in epidermalization medium for 8 days.

In some forms, the droplet of mesenchymal cell and matrix contains about 500 to about 10000 cells, about 1000 to about 5000 cells, or about 1000 to about 3000 cells, or preferably about 1250 mesenchymal cells. In some forms, the mesenchymal cell microsphere-epithelial cell mixture contains at least one or one mesenchymal cell-matrix microsphere and about 500 to about 10000, about 1000 to about 5000, or about 1000 to about 3000, or preferably about 1250 epithelial cells. In some forms, prior to forming the microsphere, the mesenchymal cells were cultured in monolayer culture for no more than 20 passages, preferably 5 passages.

In some forms, the mesenchymal cell-matrix microsphere has one or more features indicative of its hair inductivity. In some forms, the one or more features indicative of the hair inductivity of the mesenchymal cell-matrix microsphere comprises expression of alkaline phosphate, expression of versican, expression of fibronectin, activation of the Wnt signaling pathway, activation of the BMP signaling pathway, or a combination thereof. In some forms, the bioengineered hair follicle has one or more features indicative of hair inductivity. In some forms, the one or more features indicative of hair inductivity of the bioengineered hair follicle comprises expression of alkaline phosphate, expression of fibronectin, or a combination thereof.

In some forms, the bioengineered hair follicle has one or more features indicative of proliferation of epithelial cells. In some forms, the one or more features indicative of proliferation of epithelial cells comprises expression of cytokeratin, expression of Integrin α6, or a combination thereof. In some forms, the bioengineered hair follicle has one or more features indicative of hair differentiation. In some forms, the one or more features indicative of hair differentiation comprises expression of keratin 75.

In some forms, the cells in the bioengineered hair follicle have both cell-cell contacts and cell-extracellular matrix contacts. In some forms, a majority of the mesenchymal cells in the mesenchymal cell-matrix microsphere are not encased in matrix such that they do not contact another mesenchymal cell. In some forms, a majority of the mesenchymal cells in the mesenchymal cell-matrix microsphere have both cell-cell contacts and cell-extracellular matrix contacts.

In some forms, the mesenchymal cell-matrix microsphere comprises a spherical structure morphologically similar to native dermal papilla structure. In some forms, the spherical structure has a diameter ranging from 50 to 2000 μm, from 100 to 500 μm, from 50 to 500 μm, or preferably 200 to 250 μm. In some forms, the bioengineered hair follicle comprises a tubular structure morphologically similar to native hair follicles. In some forms, the tubular structure has a diameter ranging from 50 to 500 μm, or preferably from 100 to 250 μm, and a length ranging from 100 to 2000 μm, or preferably from 200 to 1000 μm.

In some forms, the mesenchymal cell-matrix microsphere is cultured in the absence of any other mesenchymal cell-matrix microsphere in the same vessel. In some forms, the vessel in which the mesenchymal cell-matrix microsphere is cultured is a single well in a multiwell plate. In some forms, other mesenchymal cell-matrix microspheres are each cultured in a different, other wells of the multiwell plate while the mesenchymal cell-matrix microsphere is cultured. In some forms, the mesenchymal cell-matrix microsphere is not removed from the vessel during the culturings until the bioengineered hair follicle is produced.

Also disclosed are bioengineered hair follicles produced by any of the methods disclosed herein.

Also disclosed are methods of using the disclosed bioengineered hair follicles. In some forms the method comprises contacting the bioengineered hair follicle with a test compound, measuring a feature of the bioengineered hair follicle, comparing the measured feature to the same feature measured in a control bioengineered hair follicle that was not contacted with the test compound, wherein a difference in the measured features indicates that the test compound affects the measured feature of the bioengineered hair follicle.

In some forms, the measured feature is hair follicle growth, wherein a difference in the measure hair follicle growth indicates that the test compound affects hair follicle growth.

Also disclosed are methods of using the disclosed bioengineered hair follicles for the prophylactic or therapeutic treatment of a state of reduced pilosity.

Also disclosed are methods of using the disclosed bioengineered hair follicles for the treatment of alopecia.

Preferred embodiments of the invention are as follows.

1. A method of producing a bioengineered hair follicle, the method comprising
forming a microsphere comprising mesenchymal cells and extracellular matrix by dispensing a droplet of a suspension of the mesenchymal cells and extracellular matrix into a vessel, and incubating the droplet in a culture vessel, thereby forming a mesenchymal cell-matrix microsphere;
culturing the mesenchymal cell-matrix microsphere in the vessel in the presence of supplementary factors;
dispensing to the vessel a droplet of a suspension of epithelial cells in close proximity to the mesenchymal cell-matrix microsphere to form a mesenchymal microsphere-epithelial cell mixture, and culturing the mesenchymal microsphere-epithelial cell mixture; and
changing the medium in the vessel to epidermalization medium and culturing, thereby producing a bioengineered hair follicle.

2. The method of embodiment 1, wherein the droplet of the suspension of the mesenchymal cells and extracellular matrix has a volume ranging from 0.5 to 10.0 μL, from 1.0 to 5.0 μL, or preferably from 2.0 to 3.0 μL.

3. The method of embodiment 1 or 2, wherein the suspension of the mesenchymal cells and extracellular matrix comprises the mesenchymal cells at a density of from 1×10⁴ to 1×10⁷ cells/ml, and extracellular matrix at a concentration of from 0.01 mg/ml to 3.0 mg/ml or 0.01 mg/ml to 2.0 mg/ml, preferably from 0.05 mg/ml to 0.5 mg/ml.

4. The method of any one of embodiments 1 to 3, wherein the mesenchymal cells are human dermal papilla cells (DPCs), human mesenchymal stem cells, human fibroblasts, or a combination thereof.

5. The method of any one of embodiments 1 to 4, wherein the extracellular matrix comprises collagen, fibronectin, fibrinogen, laminin, glycosaminoglycans, vitronectin, or a combination thereof.

6. The method of any one of embodiments 1 to 5, wherein the extracellular matrix comprises or substantially consists of collagen.

7. The method of any one of embodiments 1 to 6, wherein the culture vessel comprises a 384 well culture plate, a custom-made 88 well microwell, or a PDMS-based microwell.

8. The method of any one of embodiments 1 to 7, wherein the supplementary factors comprise FGF, HGFs, Wnt, BMP, PDGF, or a combination thereof.

9. The method of any one of embodiments 1 to 8, wherein the mesenchymal cell-matrix microsphere is cultured in the vessel in the presence of supplementary factors at from 25° C. to 39ºC, preferably 37° C. in a humidified atmosphere with from 3.5% to 6% CO₂ for from 1 to 100 hours, preferably from 12 hours to 30 hours.

10. The method of any one of embodiments 1 to 9, wherein the droplet of the suspension of the epithelial cells has a volume ranging from 0.5 to 10.0 μL, 1.0 to 5.0 μL, or preferably from 2.0 to 3.0 μL, and preferably the suspension contains the epithelial cells at a density of from 1×10⁴ to 1×10⁷ cells/ml, or preferably 1×10⁵ to 1×10⁶ cells/ml.

11. The method of any one of embodiments 1 to 10, wherein the mesenchymal microsphere-epithelial cell mixture is cultured at from 35° C. to 39ºC in a humidified atmosphere with from 3.5% to 6% CO₂ for from 1 hours to 100 hours, 5 hours to 50 hours, or preferably 18 hours to 30 hours.

12. The method of any one of embodiments 1 to 11, wherein the epithelial cells are human epidermal keratinocytes, human hair follicle keratinocytes, human epidermal progenitor or stem cells, human iPSC derived epithelial cells, or a combination thereof.

13. The method of any one of embodiments 1 to 12, wherein the ratio of mesenchymal cells to epithelial cells is from 0.1:1 to 10:1, preferably 1:1;
preferably, wherein the mesenchymal cells are human dermal papilla cells (DPCs), and the epithelial cells are human epidermal keratinocytes, and the ratio of DPC to keratinocytes is from 0.1:1 to 10:1, preferably 1:1.

14. The method of any one of embodiments 1 to 13, wherein the droplets of mesenchymal cells and extracellular matrix are incubated overnight, wherein the mesenchymal cell-matrix microsphere is cultured overnight, and wherein the mesenchymal cell microsphere-epithelial cell mixture is cultured overnight.

15. The method of any one of embodiments 1 to 14, wherein the mesenchymal cell microsphere-epithelial cell mixture is cultured in epidermalization medium for 8 days.

16. The method of any one of embodiments 1 to 15, wherein the droplet of mesenchymal cells and matrix contains about 500 to about 10000 cells, about 1000 to about 5000 cells, or about 1000 to about 3000 cells, or preferably about 1250 mesenchymal cells.

17. The method of any one of embodiments 1 to 16, wherein the mesenchymal cell microsphere-epithelial cell mixture contains at least one or one mesenchymal cell-matrix microsphere and about 500 to about 10000, about 1000 to about 5000, or about 1000 to about 3000, or preferably about 1250 epithelial cells.

18. The method of any one of embodiments 1 to 17, wherein the mesenchymal cell-matrix microsphere has one or more features indicative of its hair inductivity, preferably the one or more features indicative of the hair inductivity of the mesenchymal cell-matrix microsphere comprises expression of alkaline phosphate, expression of versican, expression of fibronectin, activation of the Wnt signaling pathway, activation of the BMP signaling pathway, or a combination thereof.

19. The method of any one of embodiments 1 to 18, wherein the bioengineered hair follicle has one or more features indicative of hair inductivity, preferably the one or more features indicative of hair inductivity of the bioengineered hair follicle comprises expression of alkaline phosphate, expression of fibronectin, or a combination thereof.

20. The method of any one of embodiments 1 to 19, wherein the bioengineered hair follicle has one or more features indicative of proliferation of epithelial cells, preferably the one or more features indicative of proliferation of epithelial cells comprises expression of cytokeratin, expression of Integrin α6, or a combination thereof.

21. The method of any one of embodiments 1 to 20, wherein the bioengineered hair follicle has one or more features indicative of hair differentiation, preferably the one or more features indicative of hair differentiation comprises expression of keratin 75.

22. The method of any one of embodiments 1 to 21, wherein the cells in the bioengineered hair follicle have both cell-cell contacts and cell-extracellular matrix contacts.

23. The method of any one of embodiments 1 to 22, wherein the mesenchymal cell-matrix microsphere comprises a spherical structure morphologically similar to native dermal papilla structure.

24. The method of embodiment 23, wherein the spherical structure has a diameter ranging from 50 to 2000 μm.

25. The method of any one of embodiments 1 to 22, wherein the bioengineered hair follicle comprises a tubular structure morphologically similar to native hair follicles.

26. The method of any one of embodiments 1 to 25, wherein the mesenchymal cell-matrix microsphere is cultured in the absence of any other mesenchymal cell-matrix microsphere in the same vessel, or in a single well in a multiwell plate.

27. A bioengineered hair follicle produced by the method of any one of embodiments 1 to 26.

28. A method of using the bioengineered hair follicle of embodiment 27, the method comprising contacting the bioengineered hair follicle with a test compound, measuring a feature of the bioengineered hair follicle, comparing the measured feature to the same feature measured in a control bioengineered hair follicle that was not contacted with the test compound, wherein a difference in the measured features indicates that the test compound affects the measured feature of the bioengineered hair follicle.

29. The method of embodiment 28, wherein the measured feature is hair follicle growth, wherein a difference in the measure hair follicle growth indicates that the test compound affects hair follicle growth.

30. A method of using the bioengineered hair follicle of embodiment 27 for the prophylactic or therapeutic treatment of a state of reduced pilosity.

31. A method of using the bioengineered hair follicle of embodiment 27 for the treatment of alopecia.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 is a graph showing the temporal change in the size of DPC microspheres as a function of collagen concentration. All data are presented as mean±SD, n=4.

FIGS. 2A-2G are graphs of gene expression of dermal papilla cells signature genes related to hair inductivity. Genes assessed were ALPL (FIG. 2A), HEY1 (FIG. 2B), BMP2 (FIG. 2C), BMP4 (FIG. 2D), NOG (FIG. 2E), LEF1 (FIG. 2F), and VCAN (FIG. 2G). All samples were normalized to levels of the reference gene GAPDH. Error bars represent the standard error of means. 4 biological triplicates×4 technical replicates in each group. Group name abbreviations: C0: DPC cell aggregates; C1: DPC-collagen microspheres at 0.1 mg/ml collagen concentration; CIF: DPC-collagen microspheres at 0.1 mg/ml collagen concentration+0.05 mg/ml fibronectin; C3: DPC-collagen microspheres at 0.3 mg/ml collagen concentration; C5: DPC-collagen microspheres at 0.5 mg/ml collagen concentration; C10: DPC-collagen microspheres at 1 mg/ml collagen concentration. * p<0.05, **p<0.01, ***p<0.001, ****p<0.0001.

FIGS. 3A-3G are graphs of dependence of gene expression levels on collagen concentration. Genes assessed were ALPL (FIG. 3A), HEY1 (FIG. 3B), BMP2 (FIG. 3C), BMP4 (FIG. 3D), NOG (FIG. 3E), LEF1 (FIG. 3F), and VCAN (FIG. 3G). All samples were normalized to levels of the reference gene GAPDH. Error bars represent the standard error of means.

FIG. 4 is a series of graphs showing cell viability in the 3D bioengineered hair follicle model. Bioengineered hair follicles were treated with 5 μM, 10 μM, 20 μM or without minoxidil for 2 days, then performed Live/Dead staining. Number of dead cells per microspheres added with varying concentration of minoxidil were quantified. Mean±SD, n=4. No statistical significance was observed in the four groups. Scale bar: 50 μm (upper panel), 200 μm (lower panel).

FIG. 5 is a series of graphs of immunofluorescence staining showing expressions of Krt75, fibronectin, and integrin α6 in bioengineered hair follicle model after treatment with 10 μM, 20 μM or without minoxidil for 4 days. Arrows indicates the proximal part of tubular structure. Scale bar: 50 μm.

FIG. 6 is a series of graphs of immunofluorescence staining showing expressions of β-catenin, BMP2 and F-actin in bioengineered hair follicle model after treatment with 5 μM, 10 μM, 20 μM or without minoxidil for 4 days. Scale bar: 50 μm.

FIG. 7 is a series of graphs showing gross appearance of nude mice after 3 weeks of subcutaneous implantation. Hair growth after 3 weeks of implantation was remarkable for experimental group (A-C) injecting with bioengineered hair follicles (DPC-HEKn microspheres) compared to control group (E-F) injecting with 2D cell suspension containing DPCs and HEKn. (A-C) In three individual experiments, hairs were generated through the skin at the area where microspheres were transplanted to. (D) Photo-crosslinked collagen membrane remained intact after 3 weeks of implantation. (E-F) No visible hairs were observed around the implantation area for control group. Insets at the top showed magnified views of the implantation area.

FIG. 8 is a series of graphs showing histological staining of nude mice dorsal skin implanted with DPC-HEKn microspheres (A-E) at 3 weeks demonstrated a number of regenerated anagen follicles while the control group (E) did not. Upper panel: Hematoxylin and Eosin (H & E) staining. Lower panel: Safranin O staining. Scale bar: 200 μm.

### DETAILED DESCRIPTION OF THE INVENTION

The disclosed method and compositions can be understood more readily by reference to the following detailed description of particular embodiments and the Example included therein and to the Figures and their previous and following description.

Bioengineering hair follicles holds a promise for hair follicle regeneration and cure of hair loss while developing a physiological relevant in vitro hair follicle model remains challenging due to easy loss of phenotypes of the hair-inductive dermal papilla cells (DPCs). Described herein are bioengineered hair follicles that recapitulate the complex in vivo environment. Collagen-DPC microspheres were first prepared then epidermal keratinocytes were added to co-culture in a defined differentiation medium for the establishment of bioengineered hair follicles. The effect of the composition of extracellular matrix in collagen-DPC microspheres on phenotype maintenance was investigated. Results show that collagen-DPC microspheres restored DP molecular signatures and were capable of inducing hair differentiation of epithelial cells. The bioengineered hair follicles demonstrated positive staining of hair-specific keratin 75 and a solid tubular structure, recapitulating at least partially the molecular signatures and morphology relevant to the in vivo hair follicle. This work thus provides a method for building bioengineered hair follicles and demonstrates the feasibility of such bioengineered hair follicles to act as a 3D in vitro hair follicle model for hair follicle research or drug screening. Such bioengineered hair follicles can also be used therapeutically and cosmetically, such as for transplantation and drug screening.

Advantageously, the method disclosed herein enables formation of individual DP in a vessel in a controllable manner. Preferably, each DP formed according to an embodiment is an independent unit without interference by surrounding cell aggregates. The formed DP is suitable for individual HF studies, drug screening or implantation purposes.

The method disclosed herein can enhance the utility rate of cells without generating by-products such as free-floating cells or tiny cell aggregates, which will uncontrollably affect the subsequent HF differentiation and the quality of the HF generated.

Moreover, the method also enables flexible adjustments on DP size meanwhile maintaining high consistency between products, well controlling the quality and yield. In particular, the method disclosed herein allows formation of DP microspheres at a dimension of from 50 to 500 μm, from 100 to 250 μm, or preferably 200 to 250 μm. In an embodiment, the DP microspheres have a dimension ranging from 200 to 250 μm which approximately that of a native human DP, and the microspheres are homogenous. The size of microspheres can be further adjusted by, including but not limited to, modifying the cell density, cell number, and/or ECM concentration. In an embodiment, the number of keratinocytes attached on DP microspheres and the different cell ratio can be well-controlled.

Advantageously, the method disclosed herein does not require genetic manipulation through reprogramming and therefore it is safer to use. Also, the method can achieve a relatively high HF differentiation efficiency, e.g. above 50%, from about 50% to 100%, from about 60% to 100%, from about 70% to 100%, from about 80% to 100%, from about 90% to 100%, or at least 90%. In an embodiment, at least 90% in particular 94% of products showed HF-like structure with a solid elongated tubular structure and demonstrated HF differentiation as indicated by hair follicle-specific marker expression.

In addition, the method enables non-HF lineage progenitor cell trans-differentiation into hair-lineage cells, which is advantageous to overcome the limited cell source due to low extraction yield of HFSCs. The method is easy-handling, time-saving and efficient. For example, it may take only one day to produce the DP and about 7 days to develop the partially differentiated HF model. In an embodiment where the method is applied with the assistance of an automated micro-dispenser system, it could produce a thousand or more bioengineered DP within a short period of time, e.g. within 10 minutes.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### Methods

Disclosed methods involving microspheres composed of mesenchymal cells and extracellular matrix, keratinocyte-containing forms of such microspheres, and bioengineered hair follicles produced from such microspheres. It has been discovered that by balancing the proportion of mesenchymal cells and extracellular matrix in the mesenchymal cell-matrix mixture, and by incubating a particular range of small volume of the mesenchymal cell-matrix mixture, mesenchymal cell-matrix microspheres can be formed that have useful properties. Most significantly, the resulting mesenchymal cell-matrix microspheres are particularly suited to be used to produce bioengineered hair follicles that have features of native hair follicles.

Disclosed are methods of producing a bioengineered hair follicle, the method comprising
forming a microsphere comprising mesenchymal cells and extracellular matrix by dispensing a droplet of a suspension of the mesenchymal cells and extracellular matrix into a vessel and incubating the droplet, thereby forming a mesenchymal cell-matrix microsphere;
culturing the mesenchymal cell-matrix microsphere in the vessel in the presence of supplementary factors;
dispensing to the vessel a droplet of a suspension of epithelial cells in close proximity to the mesenchymal cell-matrix microsphere to form a mesenchymal microsphere-epithelial cell mixture, and culturing the mesenchymal microsphere-epithelial cell mixture; and
changing the medium in the vessel to epidermalization medium and culturing, thereby producing a bioengineered hair follicle.

In some forms, and more specifically, the method can comprise forming a microsphere comprising mesenchymal cells and extracellular matrix by dispensing a droplet of a suspension of the mesenchymal cells and extracellular matrix into a vessel, and incubating the droplet at a temperature of from 25° C. to 39ºC, from 35° C. to 39° C., or preferably 37° C., in a humidified atmosphere optionally with from 3.5% to 6% CO₂ for from 1 hour to 100 hours, from 5 hours to 50 hours, or preferably from 8 hours to 30 hours, in a culture vessel, thereby forming a mesenchymal cell-matrix microsphere;
culturing the mesenchymal cell-matrix microsphere in the vessel in the presence of supplementary factors;
dispensing to the vessel a droplet of a suspension of epithelial cells in close proximity to the mesenchymal cell-matrix microsphere to form a mesenchymal microsphere-epithelial cell mixture, and culturing the mesenchymal microsphere-epithelial cell mixture; and
changing the medium in the vessel to epidermalization medium and culturing at from 35° C. to 39ºC in a humidified atmosphere optionally with from 3.5% to 6% CO₂ for from 1 to 20 days, or preferably from 3 to 10 days, thereby producing a bioengineered hair follicle.

In some forms, the droplet of the suspension of the mesenchymal cells and extracellular matrix has a volume ranging from 0.5 to 10.0 μL, from 1.0 to 5.0 μL, or preferably from 2.0 to 3.0 μL. In some forms, the suspension of the mesenchymal cells and extracellular matrix comprises the mesenchymal cells at a density of from 1×10⁴ to 1×10⁷ cells/ml, or preferably 1×10⁵ to 1×10⁶ cells/ml, and extracellular matrix at a concentration of from 0.01 mg/ml to 2.0 mg/ml, or preferably from 0.05 mg/ml to 0.5 mg/ml.

In some forms, the mesenchymal cells are human dermal papilla cells (DPCs), human mesenchymal stem cells, human fibroblasts, or a combination thereof. In some forms, the extracellular matrix comprises collagen, fibronectin, fibrinogen, laminin, glycosaminoglycans, vitronectin, or a combination thereof. In some forms, the extracellular matrix comprises or substantially consists of collagen.

In some forms, the culture vessel is comprised in a culture platform, wherein the culture platform is a 384 well culture plate, a custom-made 88 well microwell, or a PDMS-based microwell. In some forms, the supplementary factors comprise FGF, HGFs, Wnt, BMP, PDGF, or a combination thereof.

In some forms, the mesenchymal cell-matrix microsphere is cultured in the vessel in the presence of supplementary factors at from 25° ° C. to 39ºC, preferably 37° C. in a humidified atmosphere with from 3.5% to 6% CO₂ for from 1 to 100 hours, preferably from 12 hours to 30 hours.

In some forms, the droplet of the suspension of the epithelial cells has a volume ranging from 0.5 to 10.0 μL, 1.0 to 5.0 μL, or preferably from 2.0 to 3.0 μL, and preferably the suspension contains the epithelial cells at a density of from 1×10⁴ to 1×10⁷ cells/ml, or preferably 1×10⁵ to 1×10⁶ cells/ml. In some forms, the mesenchymal microsphere-epithelial cell mixture is cultured at from 35° C. to 39ºC in a humidified atmosphere with from 3.5% to 6% CO₂ for from 1 hours to 100 hours, 5 hours to 50 hours, or preferably 18 hours to 30 hours. In some forms, the epithelial cells are human epidermal keratinocytes, human hair follicle keratinocytes, human epidermal progenitor cells, human iPSC derived epithelial cells, or a combination thereof.

In some forms, all of the incubations and culturings are performed at 37° C. in a humidified atmosphere with 5% CO₂. In some forms, the droplets of mesenchymal cells and extracellular matrix are incubated overnight, wherein the mesenchymal cell-matrix microsphere is cultured overnight, and wherein the mesenchymal cell microsphere-epithelial cell mixture is cultured overnight. In some forms, the mesenchymal cell microsphere-epithelial cell mixture is cultured in epidermalization medium for 8 days.

In some forms, the droplet of mesenchymal cell and matrix contains about 500 to about 10000 cells, about 1000 to about 5000 cells, or about 1000 to about 3000 cells, or preferably about 1250 mesenchymal cells. In some forms, the mesenchymal cell microsphere-epithelial cell mixture contains at least one or one mesenchymal cell-matrix microsphere and about 500 to about 10000, about 1000 to about 5000, or about 1000 to about 3000, or preferably about 1250 epithelial cells. In some forms, prior to forming the microsphere, the mesenchymal cells were cultured in monolayer culture for no more than 20 passages, preferably 5 passages.

In some forms, the mesenchymal cell-matrix microsphere has one or more features indicative of its hair inductivity. In some forms, the one or more features indicative of the hair inductivity of the mesenchymal cell-matrix microsphere comprises expression of alkaline phosphate, expression of versican, expression of fibronectin, activation of the Wnt signaling pathway, activation of the BMP signaling pathway, or a combination thereof. In some forms, the bioengineered hair follicle has one or more features indicative of hair inductivity. In some forms, the one or more features indicative of hair inductivity of the bioengineered hair follicle comprises expression of alkaline phosphate, expression of fibronectin, or a combination thereof.

In some forms, the bioengineered hair follicle has one or more features indicative of proliferation of epithelial cells. In some forms, the one or more features indicative of proliferation of epithelial cells comprises expression of cytokeratin, expression of Integrin α6, or a combination thereof. In some forms, the bioengineered hair follicle has one or more features indicative of hair differentiation. In some forms, the one or more features indicative of hair differentiation comprises expression of keratin 75.

In some forms, the cells in the bioengineered hair follicle have both cell-cell contacts and cell-extracellular matrix contacts. In some forms, a majority of the mesenchymal cells in the mesenchymal cell-matrix microsphere are not encased in matrix such that they do not contact another mesenchymal cell. In some forms, a majority of the mesenchymal cells in the mesenchymal cell-matrix microsphere have both cell-cell contacts and cell-extracellular matrix contacts.

In some forms, the mesenchymal cell-matrix microsphere comprises a spherical structure morphologically similar to native dermal papilla structure. In some forms, the spherical structure has a diameter ranging from 50 to 2000 μm, preferably 100 to 500 μm. In some forms, the bioengineered hair follicle comprises a tubular structure morphologically similar to native hair follicles. In some forms, the tubular structure has a diameter ranging from 50 to 500 μm, or preferably from 100 to 250 μm, and a length ranging from 100 to 2000 μm, or preferably from 200 to 1000 μm.

In some forms, the mesenchymal cell-matrix microsphere is cultured in the absence of any other mesenchymal cell-matrix microsphere in the same vessel. In some forms, the vessel in which the mesenchymal cell-matrix microsphere is cultured is a single well in a multiwell plate. In some forms, other mesenchymal cell-matrix microspheres are each cultured in a different, other wells of the multiwell plate while the mesenchymal cell-matrix microsphere is cultured. In some forms, the mesenchymal cell-matrix microsphere is not removed from the vessel during the culturings until the bioengineered hair follicle is produced.

Also disclosed are methods of using the disclosed bioengineered hair follicles. In some forms the method comprises contacting the bioengineered hair follicle with a test compound, measuring a feature of the bioengineered hair follicle, comparing the measured feature to the same feature measured in a control bioengineered hair follicle that was not contacted with the test compound, wherein a difference in the measured features indicates that the test compound affects the measured feature of the bioengineered hair follicle.

In some forms, the measured feature is hair follicle growth, wherein a difference in the measure hair follicle growth indicates that the test compound affects hair follicle growth.

Also disclosed are methods of using the disclosed bioengineered hair follicles for the prophylactic or therapeutic treatment of a state of reduced pilosity.

Also disclosed are methods of using the disclosed bioengineered hair follicles for the treatment of alopecia.

The disclosed bioengineered hair follicles can be used for the production of skin equivalents. Preferably, the skin equivalents are constructed, such as by using Matriderm (Dr. Suwelack Skin & Health Care AG), according to standard methods, and the insertion sites for the bioengineered hair follicles are cut at regular intervals by means of a 2-photon laser, or pre-perforated with a punch. The disclosed bioengineered hair follicles can also be used as implants. Therefore, disclosed are implant comprising as active ingredient an effective amount of the disclosed bioengineered hair follicles, optionally together with pharmaceutically tolerable adjuvants. Similarly, the disclosed skin equivalent can be used as transplant. Thus, disclosed is a transplant comprising as active ingredient an effective amount of the disclosed skin equivalent, optionally together with pharmaceutically tolerable adjuvants.

The term “effective amount” denotes an amount of the implant or transplant, respectively, having a prophylactically or therapeutically relevant effect on a disease or pathological conditions. A prophylactic effect prevents the outbreak of a disease or even the infection with a pathogen after the infiltration of single representatives such that the subsequent propagation of the pathogen is strictly diminished, or it is even completely inactivated. A therapeutically relevant effect relieves to some extent one or more symptoms of a disease or returns to normal either partially or completely one or more physiological or biochemical parameters associated with or causative of the disease or pathological conditions. The respective amount for administering the implant or transplant, respectively, is sufficiently high in order to achieve the desired prophylactic or therapeutic effect of reducing symptoms of reduced amount of hair. It will be understood that the specific dose level, frequency and period of administration to any particular mammal will depend upon a variety of factors including the activity of the specific components employed, the age, body weight, general health, sex, diet time of administration, route of administration, drug combination, and the severity of the specific therapy. Using well-known means and methods, the exact amount can be determined by one of skill in the art as a matter of routine experimentation.

The disclosed implants or transplants are produced in a known way using common solid or liquid carriers, diluents and/or additives and usual adjuvants for pharmaceutical engineering and with an appropriate amount depending on the intended mode of application. These pharmaceutically acceptable excipients comprise salts, buffers, fillers, chelating agents, antioxidants, solvents, bonding agents, lubricants, coatings, additives, preservatives, and suspending agents. In the meaning of the invention, an “adjuvant” denotes every substance that enables, intensifies or modifies a specific body response as result of implanting or transplanting if administered simultaneously, contemporarily or sequentially. The amount of excipient material that is combined with the active ingredient to produce a single dosage form varies depending upon the host treated and the particular mode of administration.

Depending upon the manner of introduction, the implant or transplant, respectively, may be formulated in a variety of ways. The concentration of therapeutically active ingredients in the formulation may vary from about 0.1 to 100 wt %. They may be administered alone or in combination with other treatments.

The disclosed bioengineered hair follicles and/or skin equivalents are also useful for the prophylactic or therapeutic treatment of a condition of reduced amount of hair. The aforementioned products of the disclosed methods can be used for the therapeutic treatment. A therapeutically relevant effect relieves to some extent one or more symptoms of a reduced amount of hair, or returns to normality, either partially or completely, one or more physiological parameters associated with or causative of the pathological conditions. Monitoring is considered as a kind of treatment provided that the products of the inventive methods are administered in distinct intervals, e.g., in order to booster the proliferation response and eradicate the symptoms of the condition completely. Either identical products or different products can be applied. Prophylactic treatment is generally advisable if the subject possesses any preconditions for the beginning of hair loss, such as a familial disposition, a genetic defect, or a previously passed disease.

The pathologic conditions of a reduced amount of hair may be the result of alopecia (e.g., androgenetic alopecia, alopecia greata, etc.), hereditary baldness, scarring, burns, radiation therapy, chemotherapy, disease-related loss of hair, accidental injury, damage to hair follicle, surgical trauma, an incisional wound, or a donor site wound from a skin transplant.

The disclosed bioengineered hair follicles and/or skin equivalents can be used for the production of an implant or transplant, respectively, for the prophylactic or therapeutic treatment of a condition of reduced amount of hair. The implant and transplant can be either administered to prevent the initiation of hair loss of a mammal, preferably a human individual, and the resulting trouble in advance, or to treat the arising and continuing symptoms.

Also disclosed are methods for treating a condition of reduced amount of hair, wherein the bioengineered hair follicles and/or skin equivalents are incorporated into the skin of a mammal in need of such treatment. The bioengineered hair follicles, especially autologous/allogenic bioengineered hair follicles, can be used for implantation with the aim of inducing hair growth, whereas the skin substitutes do regenerate skin, preferably the scalp. The bioengineered hair follicles are incorporated into the openings of previously depilated, miniaturized hair follicles (isthmus) of affected skin areas. Preferably, the bioengineered hair follicles are injected, more preferably by means of a specially constructed device of about 150 μm in size. It is also preferred that all components are used in an autologous fashion and treated under GLP/GMP conditions.

The bioengineered hair follicles stimulate the new development of hair growth, such as in cases of hereditary baldness, scarring (burns), disease-related loss of hair, chemotherapy/radiation-induced loss of hair, and the like. The bioengineered hair follicles can also be used for the direct pharmacological and cosmetic in vitro testing of substances, which exert a hair-modulating influence. The hair-modulating effects are especially selected from the group of hair growth, hair shape, hair structure, hair color, and hair pigmentation. It is preferred to analyze the effect of modifying hair growth—with the intention of promoting hair growth in cases of hair loss, such as caused by alopecia, as well as inhibiting hair growth in cases of excessive, undesirable hair growth, such as caused by hypertrichosis and/or hirsutism, or female beard growth, or undesirable body hair. In particular, the use of a high-throughput method allows the pharmaceutical and cosmetic industries to effectively test existing or new substances for a potential hair growth-modulating effect. The substances comprise pharmaceutical agents, cosmetic agents, chemical compounds, polymeric compounds, growth factors, cellular products, living cells and/or biomolecules. Furthermore, when adding melanocytes, i.e., the pigment-forming cells, to the bioengineered hair follicles, it is possible to investigate substance effects on the pigmentation and/or coloring of the hair shaft being formed. Likewise, the substance effect on hair shape and hair structure can be tested, e.g., formation of curls, etc.

The following end points can be evaluated or measured to obtain information on the effectiveness of substances in regard to an improvement in hair structure and the influencing of hair growth: analysis of hair shaft formation, length growth and characteristics of the hair shaft, hair array analysis, volume and structure of the dermal papilla, proliferation measurement (e.g. Ki67 expression, BrdU incorporation, etc.), apoptosis measurement (e.g. TUNEL, enzyme assays, annexin measurement, etc.), differential marker analysis (e.g. immunohistology, in situ hybridization, RT-PCR, etc.), determination of alkaline phosphatase as DPF marker, analysis of certain hair-specific proteins (e.g. hair-specific keratins, etc.), analysis of cytokines, growth factors, chemokines and all kinds of messenger substances formed inter alia by the dermal papilla (e.g. by BioPlex, ELISA, etc.), and/or proteome or expression analysis of matrix proteins, growth factors (e.g. MSP, HGF, CTGF, etc.), transcription factors, molecules of the wnt-pathway (e.g. DKK1, BMP2-4, etc.), interleukins (e.g. IL-6, etc.) and/or chemokines/chemokine receptors (e.g. CXCR, etc.), which exhibit an enhanced appearance, as well as apoptosis-inducing molecules and/or proliferation-stimulating molecules, which exhibit a reduced appearance. The influence on hair pigmentation can be measured by means of arrangement/migration of melanocytes, melanin granula formation/distribution, and the activity of tyrosinase and/or array analysis of gene expression involved in melanin synthesis. Other embodiments, modifications and variations of the present invention will be readily apparent to the expert on reading the specification and can be put into practice without departing from the scope of the invention.

Furthermore, the bioengineered hair follicles can be used separately, or in connection with the generation of skin equivalents with hair follicles, for the pharmacological and toxicological in vitro testing of substances in medicine, pharmacy, and beauty culture. Such use, e.g., performed as high-throughput method, is of special interest for the pharmaceutical, chemical and cosmetic industries if obliged to test their substances and products for toxic effects. For replacement of animal tests by suitable in vitro test methods, the bioengineered hair follicles themselves, but also, artificial skin replacement systems with integrated bioengineered hair follicles can be employed as ideal screening systems for toxicological investigations including irritations, genotoxic effects, etc. The disclosed bioengineered hair follicles may completely replace animal tests, as well as substitute less suitable in vitro models being currently available, since the present models make the analysis of complex physiological processes possible. Such tests can be performed by exposing the disclosed bioengineered hair follicles to a substance of interest in a bioreactor. Following a substance-specific incubation period, which is particularly between 3 minutes and 4 hours, the bioengineered hair follicle is washed with medium, and subsequently analyzed by suitable assays exemplarily described in the prior course of the specification.

Also disclosed are methods for screening substances, which modulate hair properties, comprising the steps of providing bioengineered hair follicles, incubating at least one of the bioengineered hair follicles with substances to be screened, and comparing parameters of hair properties in the bioengineered hair follicles with another bioengineered hair follicle that is not incubated with the substances. The method makes the identification and analysis of substances possible, which exert an influence on hair via the bioengineered hair follicles. At least two subsets of bioengineered hair follicles are provided; one is used for screening while the other one serves as negative control. Preferably, the number of screening parts exceeds the number of control parts. Usually, numerous bioengineered hair follicles are subjected to a high-throughput screening. The substances to be screened are not restricted anyway. In some forms, the substances are selected from the group of nucleic acids including RNAi, rybozymes, aptamers, antibodies, peptides, carbohydrates, polymers, small molecules having a molecular weight between 50 and 1,000 Da, and proteins, preferably antibodies, cytokines and lipocalins. These substances are often available in libraries. It is preferred to incubate a single compound on a bioengineered hair follicle. However, it is also possible to investigate the cooperative effect of substances by incubating at least two substances on a bioengineered hair follicle. A further subset of bioengineered hair follicles is simultaneously incubated in the absence of the substances. The incubation process depends on various parameters, e.g., the cell types and the sensitivity of detection, which optimization follows routine procedures known to those skilled in the art. The identification of effective substances is indirectly performed, preferably by determining the expression patterns and/or the cell viability, which are altered. The determination is performed at a specified moment and correlated to the signal strength at the beginning of the experiment and the negative control. Suitable tests are known to those skilled in the art or can be easily designed as a matter of routine.

Also disclosed are kits comprising the bioengineered hair follicles, skin equivalent, implant, and/or transplant, particularly in order to perform the disclosed methods of treating a condition of reduced amount of hair, or screening substances, respectively. The kit may include an article that comprises written instructions, or directs the user to written instructions for how to practice the methods. For further details, reference may be made to the foregoing observations on the treatment methods as well as the screening methods, which also apply accordingly to the kit.

The term “droplet” refers to a small volume of liquid (e.g., <100 μL). In the context of the disclosed methods, droplet refers to such a small volume of aqueous solution and/pr suspension. Preferably, the size of such a droplet is such that it forms a droplet on a sufficiently hydrophobic surface.

A “culture platform” refers to a material or structure on which or in which a droplet contain cells can be incubated or cultured. Examples of culture platforms include but are not limited to parafilm, coverglasses, slides, watchglasses, petri dishes, and multiwell plates.

The term “vessel” refers to a separate portion of a well-less culture platform or to a single well of a single or multiwell culture platform.

The term “hit” refers to a test compound that shows desired properties in an assay. The term “test compound” refers to a chemical to be tested by one or more screening method(s) as a putative modulator. A test compound can be any chemical, such as an inorganic chemical, an organic chemical, a protein, a peptide, a carbohydrate, a lipid, or a combination thereof. Usually, various predetermined concentrations of test compounds are used for screening, such as 0.01 micromolar, 1 micromolar and 10 micromolar. Test compound controls can include the measurement of a signal in the absence of the test compound or comparison to a compound known to modulate the target.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “inhibit” means to reduce or decrease in activity or expression. This can be a complete inhibition of activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “monitoring” as used herein refers to any method in the art by which an activity can be measured.

The term “providing” as used herein refers to any means of adding a compound, molecule, or composition to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The term “preventing” as used herein refers to administering a compound or composition prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the disclosed bioengineered hair follicles.

As used herein, “subject” includes, but is not limited to, animals, more specifically a mammal (e.g., a human, horse, pig, rabbit, dog, sheep, goat, non-human primate, cow, cat, guinea pig or rodent). A patient refers to a subject afflicted with a condition, disease, or disorder. The term “patient” includes human and veterinary subjects.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

A cell can be in vitro. Alternatively, a cell can be in vivo and can be found in a subject. A “cell” can be a cell from any organism including, but not limited to, a bacterium.

By “pharmaceutically acceptable” is meant a material that is not biologically or otherwise undesirable, i.e., the material can be administered to a subject along with the selected compound or composition without causing any undesirable biological effects or interacting in a deleterious manner with any of the other components of the pharmaceutical composition in which it is contained.

### Materials

Disclosed are microspheres composed of mesenchymal cells and extracellular matrix, epithelial cell-containing forms of such microspheres, and bioengineered hair follicles produced from such microspheres.

In some forms, the mesenchymal cells are human dermal papilla cells (DPCs), human mesenchymal stem cells, human fibroblasts, or a combination thereof. In some forms, the extracellular matrix comprises collagen, fibronectin, fibrinogen, laminin, glycosaminoglycans, vitronectin, or a combination thereof. In some forms, the extracellular matrix comprises or substantially consists of collagen. In some forms, the epithelial cells are human epidermal keratinocytes, human hair follicle keratinocytes, human epidermal progenitor cells, human iPSC derived epithelial cells, or a combination thereof.

In some forms, the mesenchymal cell-matrix microsphere has one or more features indicative of its hair inductivity. In some forms, the one or more features indicative of the hair inductivity of the mesenchymal cell-matrix microsphere comprises expression of alkaline phosphate, expression of versican, expression of fibronectin, activation of the Wnt signaling pathway, activation of the BMP signaling pathway, or a combination thereof. In some forms, the bioengineered hair follicle has one or more features indicative of hair inductivity. In some forms, the one or more features indicative of hair inductivity of the bioengineered hair follicle comprises expression of alkaline phosphate, expression of fibronectin, or a combination thereof.

In some forms, the bioengineered hair follicle has one or more features indicative of proliferation of epithelial cells. In some forms, the one or more features indicative of proliferation of epithelial cells comprises expression of cytokeratin, expression of Integrin α6, or a combination thereof. In some forms, the bioengineered hair follicle has one or more features indicative of hair differentiation. In some forms, the one or more features indicative of hair differentiation comprises expression of keratin 75.

In some forms, the cells in the bioengineered hair follicle have both cell-cell contacts and cell-extracellular matrix contacts. In some forms, a majority of the mesenchymal cells in the mesenchymal cell-matrix microsphere are not encased in matrix such that they do not contact another mesenchymal cell. In some forms, a majority of the mesenchymal cells in the mesenchymal cell-matrix microsphere have both cell-cell contacts and cell-extracellular matrix contacts.

In some forms, the mesenchymal cell-matrix microsphere comprises a spherical structure morphologically similar to native dermal papilla structure. In some forms, the spherical structure has a diameter ranging from 50 to 2000 μm, preferably 100 to 500 μm. In some forms, the bioengineered hair follicle comprises a tubular structure morphologically similar to native hair follicles. In some forms, the tubular structure has a diameter ranging from 50 to 500 μm, or preferably from 100 to 250 μm, and a length ranging from 100 to 2000 μm, or preferably from 200 to 1000 μm.

Preferably, the disclosed mesenchymal cell-matrix microspheres, epithelial cell-containing forms of such microspheres, and bioengineered hair follicles are produced by forming a microsphere comprising mesenchymal cells and extracellular matrix by dispensing a droplet of a suspension of the mesenchymal cells and extracellular matrix into a vessel and incubating the droplet, thereby forming a mesenchymal cell-matrix microsphere;
culturing the mesenchymal cell-matrix microsphere in the vessel in the presence of supplementary factors;
dispensing to the vessel a droplet of a suspension of epithelial cells in close proximity to the mesenchymal cell-matrix microsphere to form a mesenchymal microsphere-epithelial cell mixture, and culturing the mesenchymal microsphere-epithelial cell mixture; and
changing the medium in the vessel to epidermalization medium and culturing, thereby producing a bioengineered hair follicle.

In some forms, and more specifically, the disclosed mesenchymal cell-matrix microspheres, epithelial cell-containing forms of such microspheres, and bioengineered hair follicles are produced by forming a microsphere comprising mesenchymal cells and extracellular matrix by dispensing a droplet of a suspension of the mesenchymal cells and extracellular matrix into a vessel, and incubating the droplet at a temperature of from 25° C. to 39° ° C., from 35° ° C. to 39º° C., or preferably 37° C., in a humidified atmosphere optionally with from 3.5% to 6% CO₂ for from 1 hour to 100 hours, from 5 hours to 50 hours, or preferably from 8 hours to 30 hours, in a culture vessel, thereby forming a mesenchymal cell-matrix microsphere;
culturing the mesenchymal cell-matrix microsphere in the vessel in the presence of supplementary factors;
dispensing to the vessel a droplet of a suspension of epithelial cells in close proximity to the mesenchymal cell-matrix microsphere to form a mesenchymal microsphere-epithelial cell mixture, and culturing the mesenchymal microsphere-epithelial cell mixture; and
changing the medium in the vessel to epidermalization medium and culturing at from 35° C. to 39ºC in a humidified atmosphere optionally with from 3.5% to 6% CO₂ for from 1 to 20 days, or preferably from 3 to 10 days, thereby producing a bioengineered hair follicle.

In some forms of the method of producing the disclosed mesenchymal cell-matrix microspheres, epithelial cell-containing forms of such microspheres, and bioengineered hair follicles, the droplet of the suspension of the mesenchymal cells and extracellular matrix has a volume ranging from 0.5 to 10.0 μL, from 1.0 to 5.0 μL, or preferably from 2.0 to 3.0 μL. In some forms of the method of producing the disclosed mesenchymal cell-matrix microspheres, epithelial cell-containing forms of such microspheres, and bioengineered hair follicles, the suspension of the mesenchymal cells and extracellular matrix comprises the mesenchymal cells at a density of from 1×10⁴ to 1×10⁷ cells/ml, or preferably 1×10⁵ to 1×10⁶ cells/ml, and extracellular matrix at a concentration of from 0.01 mg/ml to 2.0 mg/ml, or preferably from 0.05 mg/ml to 0.5 mg/ml.

In some forms of the method of producing the disclosed mesenchymal cell-matrix microspheres, epithelial cell-containing forms of such microspheres, and bioengineered hair follicles, the culture vessel is comprised in a culture platform, wherein the culture platform is a 384 well culture plate, a custom-made 88 well microwell, or a PDMS-based microwell. In some forms of the method of producing the disclosed mesenchymal cell-matrix microspheres, epithelial cell-containing forms of such microspheres, and bioengineered hair follicles, the supplementary factors comprise FGF, HGFs, Wnt, BMP, PDGF, or a combination thereof.

In some forms of the method of producing the disclosed mesenchymal cell-matrix microspheres, epithelial cell-containing forms of such microspheres, and bioengineered hair follicles, the mesenchymal cell-matrix microsphere is cultured in the vessel in the presence of supplementary factors at from 25° C. to 39ºC, preferably 37° C. in a humidified atmosphere with from 3.5% to 6% CO₂ for from 1 to 100 hours, preferably from 12 hours to 30 hours.

In some forms of the method of producing the disclosed mesenchymal cell-matrix microspheres, epithelial cell-containing forms of such microspheres, and bioengineered hair follicles, the droplet of the suspension of the epithelial cells has a volume ranging from 0.5 to 10.0 μL, 1.0 to 5.0 μL, or preferably from 2.0 to 3.0 μL, and preferably the suspension contains the epithelial cells at a density of from 1×10⁴ to 1×10⁷ cells/ml, or preferably 1×10⁵ to 1×10⁶ cells/ml. In some forms of the method of producing the disclosed mesenchymal cell-matrix microspheres, epithelial cell-containing forms of such microspheres, and bioengineered hair follicles, the mesenchymal microsphere-epithelial cell mixture is cultured at from 35° C. to 39ºC in a humidified atmosphere with from 3.5% to 6% CO₂ for from 1 hours to 100 hours, 5 hours to 50 hours, or preferably 18 hours to 30 hours.

In some forms of the method of producing the disclosed mesenchymal cell-matrix microspheres, epithelial cell-containing forms of such microspheres, and bioengineered hair follicles, all of the incubations and culturings are performed at 37° C. in a humidified atmosphere with 5% CO₂. In some forms of the method of producing the disclosed mesenchymal cell-matrix microspheres, epithelial cell-containing forms of such microspheres, and bioengineered hair follicles, the droplets of mesenchymal cells and extracellular matrix are incubated overnight, wherein the mesenchymal cell-matrix microsphere is cultured overnight, and wherein the mesenchymal cell microsphere-epithelial cell mixture is cultured overnight. In some forms of the method of producing the disclosed mesenchymal cell-matrix microspheres, epithelial cell-containing forms of such microspheres, and bioengineered hair follicles, the mesenchymal cell microsphere-epithelial cell mixture is cultured in epidermalization medium for 8 days.

In some forms of the method of producing the disclosed mesenchymal cell-matrix microspheres, epithelial cell-containing forms of such microspheres, and bioengineered hair follicles, the droplet of mesenchymal cell and matrix contains about 500 to about 10000 cells, about 1000 to about 5000 cells, or about 1000 to about 3000 cells, or preferably about 1250 mesenchymal cells. In some forms of the method of producing the disclosed mesenchymal cell-matrix microspheres, epithelial cell-containing forms of such microspheres, and bioengineered hair follicles, the mesenchymal cell microsphere-epithelial cell mixture contains at least one or one mesenchymal cell-matrix microsphere and about 500 to about 10000, about 1000 to about 5000, or about 1000 to about 3000, or preferably about 1250 epithelial cells. In some forms of the method of producing the disclosed mesenchymal cell-matrix microspheres, epithelial cell-containing forms of such microspheres, and bioengineered hair follicles, prior to forming the microsphere, the mesenchymal cells were cultured in monolayer culture for no more than 20 passages, preferably 5 passages.

In some forms of the method of producing the disclosed mesenchymal cell-matrix microspheres, epithelial cell-containing forms of such microspheres, and bioengineered hair follicles, the mesenchymal cell-matrix microsphere is cultured in the absence of any other mesenchymal cell-matrix microsphere in the same vessel. In some forms of the method of producing the disclosed mesenchymal cell-matrix microspheres, epithelial cell-containing forms of such microspheres, and bioengineered hair follicles, the vessel in which the mesenchymal cell-matrix microsphere is cultured is a single well in a multiwell plate. In some forms of the method of producing the disclosed mesenchymal cell-matrix microspheres, epithelial cell-containing forms of such microspheres, and bioengineered hair follicles, other mesenchymal cell-matrix microspheres are each cultured in a different, other wells of the multiwell plate while the mesenchymal cell-matrix microsphere is cultured. In some forms of the method of producing the disclosed mesenchymal cell-matrix microspheres, epithelial cell-containing forms of such microspheres, and bioengineered hair follicles, the mesenchymal cell-matrix microsphere is not removed from the vessel during the culturings until the bioengineered hair follicle is produced.

Disclosed are mixtures formed by performing or preparing to perform the disclosed method. For example, disclosed are mixtures comprising bioengineered hair follicles.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

The disclosed compositions and methods can be further understood through the following numbered paragraphs.

1. A method of producing a bioengineered hair follicle, the method comprising
forming a microsphere comprising mesenchymal cells and extracellular matrix by dispensing a droplet of a suspension of the mesenchymal cells and extracellular matrix into a vessel, and incubating the droplet at a temperature of from 25° C. to 39ºC, from 35° C. to 39ºC, or preferably 37° C., in a humidified atmosphere optionally with from 3.5% to 6% CO₂ for from 1 hour to 100 hours, from 5 hours to 50 hours, or preferably from 8 hours to 30 hours, in a culture vessel, thereby forming a mesenchymal cell-matrix microsphere;
culturing the mesenchymal cell-matrix microsphere in the vessel in the presence of supplementary factors;
dispensing to the vessel a droplet of a suspension of epithelial cells in close proximity to the mesenchymal cell-matrix microsphere to form a mesenchymal microsphere-epithelial cell mixture, and culturing the mesenchymal microsphere-epithelial cell mixture; and
changing the medium in the vessel to epidermalization medium and culturing at from 35° C. to 39° C. in a humidified atmosphere optionally with from 3.5% to 6% CO₂ for from 1 to 20 days, or preferably from 3 to 10 days, thereby producing a bioengineered hair follicle.

2. The method of paragraph 1, wherein the droplet of the suspension of the mesenchymal cells and extracellular matrix has a volume ranging from 0.5 to 10.0 μL, from 1.0 to 5.0 μL, or preferably from 2.0 to 3.0 μL.

3. The method of paragraph 1 or 2, wherein the suspension of the mesenchymal cells and extracellular matrix comprises the mesenchymal cells at a density of from 1×10⁴ to 1×10⁷ cells/ml, or preferably 1×10⁵ to 1×10⁶ cells/ml, and extracellular matrix at a concentration of from 0.01 mg/ml to 2.0 mg/ml, or preferably from 0.05 mg/ml to 0.5 mg/ml.

4. The method of any one of paragraphs 1 to 3, wherein the mesenchymal cells are human dermal papilla cells (DPCs), human mesenchymal stem cells, human fibroblasts, or a combination thereof.

5. The method of any one of paragraphs 1 to 4, wherein the extracellular matrix comprises collagen, fibronectin, fibrinogen, laminin, glycosaminoglycans, vitronectin, or a combination thereof.

6. The method of any one of paragraphs 1 to 5, wherein the extracellular matrix comprises or substantially consists of collagen.

7. The method of any one of paragraphs 1 to 6, wherein the culture vessel is comprised in a culture platform, wherein the culture platform is a 384 well culture plate, a custom-made 88 well microwell, or a PDMS-based microwell.

8. The method of any one of paragraphs 1 to 7, wherein the supplementary factors comprise FGF, HGFs, Wnt, BMP, PDGF, or a combination thereof.

9. The method of any one of paragraphs 1 to 8, wherein the mesenchymal cell-matrix microsphere is cultured in the vessel in the presence of supplementary factors at from 25° C. to 39ºC, preferably 37° C. in a humidified atmosphere with from 3.5% to 6% CO₂ for from 1 to 100 hours, preferably from 12 hours to 30 hours.

10. The method of any one of paragraphs 1 to 9, wherein the droplet of the suspension of the epithelial cells has a volume ranging from 0.5 to 10.0 μL, 1.0 to 5.0 μL, or preferably from 2.0 to 3.0 μL, and preferably the suspension contains the epithelial cells at a density of from 1×10⁴ to 1×10⁷ cells/ml, or preferably 1×10⁵ to 1×10⁶ cells/ml.

11. The method of any one of paragraphs 1 to 10, wherein the mesenchymal microsphere-epithelial cell mixture is cultured at from 35° C. to 39° C. in a humidified atmosphere with from 3.5% to 6% CO₂ for from 1 hours to 100 hours, 5 hours to 50 hours, or preferably 18 hours to 30 hours.

12. The method of any one of paragraphs 1 to 11, wherein the epithelial cells are human epidermal keratinocytes, human hair follicle keratinocytes, human epidermal progenitor cells, human iPSC derived epithelial cells, or a combination thereof.

13. The method of any one of paragraphs 1 to 12, wherein all of the incubations and culturings are performed at 37° ° C. in a humidified atmosphere with 5% CO₂.

14. The method of any one of paragraphs 1 to 13, wherein the droplets of mesenchymal cells and extracellular matrix are incubated overnight, wherein the mesenchymal cell-matrix microsphere is cultured overnight, and wherein the mesenchymal cell microsphere-epithelial cell mixture is cultured overnight.

15. The method of any one of paragraphs 1 to 14, wherein the mesenchymal cell microsphere-epithelial cell mixture is cultured in epidermalization medium for 8 days.

16. The method of any one of paragraphs 1 to 15, wherein the droplet of mesenchymal cell and matrix contains about 500 to about 10000 cells, about 1000 to about 5000 cells, or about 1000 to about 3000 cells, or preferably about 1250 mesenchymal cells.

17. The method of any one of paragraphs 1 to 16, wherein the mesenchymal cell microsphere-epithelial cell mixture contains at least one or one mesenchymal cell-matrix microsphere and about 500 to about 10000, about 1000 to about 5000, or about 1000 to about 3000, or preferably about 1250 epithelial cells.

18. The method of any one of paragraphs 1 to 17, wherein, prior to forming the microsphere, the mesenchymal cells were cultured in monolayer culture for no more than 20 passages, preferably 5 passages.

19. The method of any one of paragraphs 1 to 18, wherein the mesenchymal cell-matrix microsphere has one or more features indicative of its hair inductivity.

20. The method of paragraph 19, wherein the one or more features indicative of the hair inductivity of the mesenchymal cell-matrix microsphere comprises expression of alkaline phosphate, expression of versican, expression of fibronectin, activation of the Wnt signaling pathway, activation of the BMP signaling pathway, or a combination thereof.

21. The method of any one of paragraphs 1 to 20, wherein the bioengineered hair follicle has one or more features indicative of hair inductivity.

22. The method of paragraph 21, wherein the one or more features indicative of hair inductivity of the bioengineered hair follicle comprises expression of alkaline phosphate, expression of fibronectin, or a combination thereof.

23. The method of any one of paragraphs 1 to 22, wherein the bioengineered hair follicle has one or more features indicative of proliferation of epithelial cells.

24. The method of paragraph 23, wherein the one or more features indicative of proliferation of epithelial cells comprises expression of cytokeratin, expression of Integrin α6, or a combination thereof.

25. The method of any one of paragraphs 1 to 24, wherein the bioengineered hair follicle has one or more features indicative of hair differentiation.

26. The method of paragraph 25, wherein the one or more features indicative of hair differentiation comprises expression of keratin 75.

27. The method of any one of paragraphs 1 to 26, wherein the cells in the bioengineered hair follicle have both cell-cell contacts and cell-extracellular matrix contacts.

28. The method of any one of paragraphs 1 to 27, wherein the mesenchymal cell-matrix microsphere comprises a spherical structure morphologically similar to native dermal papilla structure.

29. The method of paragraph 28, wherein the spherical structure has a diameter ranging from 50 to 2000 μm, preferably 100 to 500 μm.

30. The method of any one of paragraphs 1 to 29, wherein the bioengineered hair follicle comprises a tubular structure morphologically similar to native hair follicles.

31. The method of paragraph 30, wherein the tubular structure has a diameter ranging from 50 to 500 μm, or preferably from 100 to 250 μm, and a length ranging from 100 to 2000 μm, or preferably from 200 to 1000 μm.

32. The method of any one of paragraphs 1 to 31, wherein the mesenchymal cell-matrix microsphere is cultured in the absence of any other mesenchymal cell-matrix microsphere in the same vessel.

33. A bioengineered hair follicle produced by the method of any one of paragraphs 1 to 32.

34. A method of using the bioengineered hair follicle of paragraph 33, the method comprising contacting the bioengineered hair follicle with a test compound, measuring a feature of the bioengineered hair follicle, comparing the measured feature to the same feature measured in a control bioengineered hair follicle that was not contacted with the test compound, wherein a difference in the measured features indicates that the test compound affects the measured feature of the bioengineered hair follicle.

35. The method of paragraph 34, wherein the measured feature is hair follicle growth, wherein a difference in the measure hair follicle growth indicates that the test compound affects hair follicle growth.

36. A method of using the bioengineered hair follicle of paragraph 33 for the prophylactic or therapeutic treatment of a state of reduced pilosity.

37. A method of using the bioengineered hair follicle of paragraph 33 for the treatment of alopecia.

### EXAMPLES

### Example 1: Development and Demonstration of Properties of Bioengineered Hair Follicles

In the present study, we developed a method for preparing bioengineered hair follicles comprising of collagen-DPC microspheres and epithelial cell populations, which represented the mesenchymal and epithelial components of the native hair follicle and reconstituted the mesenchymal-epithelial interactions. We realized that the collagen-DPC microspheres could better maintain the phenotypes and hair inductive properties of dermal papilla cells, and that the microspheres could also be used for reconstitution of bioengineered hair follicles to instruct epithelial cells differentiated into hair lineages.

Bioengineering hair follicles holds a promise for hair follicle regeneration and cure of hair loss while developing a physiological relevant in vitro hair follicle model remains challenging due to easy loss of phenotypes of the hair-inductive dermal papilla cells (DPCs). In this study, bioengineered hair follicles that recapitulate the complex in vivo environment were developed. Collagen-DPC microspheres were first prepared then epidermal keratinocytes were added to co-culture in a defined differentiation medium for the establishment of bioengineered hair follicles. The effect of the composition of extracellular matrix in collagen-DPC microspheres on phenotype maintenance was investigated. Results show that collagen-DPC microspheres restored DP molecular signatures and were capable of inducing hair differentiation of epithelial cells. The bioengineered hair follicles demonstrated positive staining of hair-specific keratin 75 and a solid tubular structure, recapitulating at least partially the molecular signatures and morphology relevant to the in vivo hair follicle. This work thus provides a method for building bioengineered hair follicles and demonstrates the feasibility of such bioengineered hair follicles to act as a 3D in vitro hair follicle model for hair follicle research or drug screening. Such bioengineered hair follicles can also be used therapeutically and cosmetically, such as for transplantation and drug screening.

### Materials and Methods

### Culture of Human DPC's and Human Epidermal Keratinocytes

Human hair dermal papilla cells (Cat. 2400, ScienCell) were cultured in gelatin-coated culture flasks with mesenchymal stem cell medium (Cat. 7501, ScienCell) consisting of 5% fetal bovine serum (FBS), 1% mesenchymal stem cell growth supplement, and 1% penicillin/streptomycin solution. Neonatal human epidermal keratinocytes (HEKn, C-0015C, Gibco) were cultured in EpiLife® medium supplemented with human keratinocyte growth supplement (HKGS, S-0015, Gibco). Cultures were maintained at 37° C. in a humidified atmosphere with 5% CO₂ and the medium was changed every other day for all cell types. Both cells were subcultured to passage 5 for microsphere fabrication.

### Preparation of Collagen-DPC Microspheres

Dermal papilla cell suspensions were mixed with neutralized rat tail type I collagen (BD Bioscience) to make cell-matrix mixtures with a cell density of 5×10⁵ cells/ml and varying collagen concentration (0.1, 0.3, 0.5, or 1.0 mg/ml) in an ice-bath. The recipe of the cell-matrix mixtures can be changed by mixing different extracellular matrix proteins accordingly, such as fibronectin and glycosaminoglycan (GAG). Droplets of the cell-matrix mixtures of 2.5 μL were dispensed into a non-adhesive Petri dish or 6-well plate culture dish covered with UV-irradiated parafilm at the bottom and were incubated at 37° C. in a humidified atmosphere with 5% CO₂ overnight to allow the formation of microspheres. Formed collagen-DPC microspheres were gently flushed with full medium and maintained free-floating in suspensions. For the individual culture of collagen-DPC microspheres, cell-matrix mixtures were added onto a coverglass with a home-made silicone biochip isolator, where the glass is pre-coated with anti-adhesive reagents such as 1% Pluronic® F127 overnight. DPC microsphere was formed first and then bring keratinocytes or progenitor cell or stem cell into close proximity to the DPC-microsphere to form the DPC-microsphere-Keratinocyte mixture at certain DPC:Keratinocyte ratio and then co-culture.

### Preparation of Bioengineered HE's

Coverglasses were coated with 1% Pluronic® F127 one day before use. DPC-matrix mixtures of 2.5 μl containing 1250 cells per droplet were dispensed into the microwell and were incubated at 37° C. in a humidified atmosphere with 5% CO₂ overnight to allow the formation of microspheres. After 24 hours, 2.5 μl of human epidermal keratinocytes suspension containing 1250 cells per droplet was added to the microwell and co-cultured with the previously formed collagen-DPC microspheres. One day after the co-culture, the medium was changed to epidermalization medium and replaced every other day. Epidermalization medium was prepared according to previously reported protocol with some minor modifications (Gangatirkar et al., 2007). The bioengineered HFs were cultured for 8 days and then used for immunofluorescence analysis.

### Immunofluorescence Analysis

Samples were fixed with 4% paraformaldehyde (Sigma-Aldrich) for 20 minutes, followed by immersion in 30% sucrose (Sigma-Aldrich) overnight at 4° C., and then embedded in OCT solution for cryosectioning. Cross-sections of 15 μm were prepared. Cryosectioned samples were rinsed with phosphate-buffered saline (PBS), permeabilized with 0.1% Triton (Roche Applied Science) for 10 minutes, washed with PBS three times, and blocked with 3% bovine serum albumin (BSA) for 30 minutes at room temperature. Samples were incubated with appropriate primary antibodies diluted in blocking buffer at 4ºC overnight, washed with PBS three times, and incubated with fluorophore-conjugated secondary antibodies for 1 hour. The following primary antibodies were used: rabbit anti-alkaline phosphatase (ALP) (1:50, ab65834, Abcam), rabbit anti-versican (1:200, PA1-1748A, Invitrogen), mouse anti-fibronectin (1:200, sc8422, Santa Cruz), mouse anti-BMP2 (1:100, ab6285, Abcam), rabbit anti-β catenin (1:150, ab16051, Abcam), mouse anti-cytokeratin 5 (1:250, MA5-12596, Invitrogen), rabbit anti-KRT 75 (1:50, PA5-67414, Invitrogen), rat anti-integrin α6 (1:100, ab105669, Abcam). Rhodamine-tagged phalloidin (1:100, R415, Molecular Probes, Life Technology) was used to label F-actin. The secondary antibodies used included Alexa Fluor 488 goat anti-mouse (A11029), Alexa Fluor 546 goat anti-rat (A11081), and Alexa Fluor 647 goat anti-rabbit (A32733, Invitrogen), were all from Molecular Probes and were all used at a dilution of 1:400. Sections were mounted with Fluoro-gel II mounting medium with DAPI (17985-50, Electron Microscopy Sciences). Images were taken using a confocal laser scanning microscope (LSM710, Carl-Zeiss; Leica TCS SP8, Leica Microsystems).

### Real-Time PCR and Statistical Analysis

Real-time PCR was performed to analyze the gene expression level of dermal papilla cells signature genes related to hair inductivity, including ALPL, HEY1, BMP2, BMP4, NOG, LEF1, and VCAN. Collagen-DPC microspheres were collected three days after culture. The total RNA was extracted using the RNeasy Mini Kit (74106, Qiagen) according to the manufacturer's instructions. RNA concentration was determined using a NanoDrop™ 2000 spectrophotometer (NanoDrop, DE, USA) and was transcribed into cDNA using High Capacity cDNA Reverse Transcription kit (Applied Biosystems).

Real-time PCR was performed using Power SYBR® Green PCR Master Mix (Applied Biosystems) under standard thermal conditions. The PCR reactions were run in Applied Biosystems 7300 Real-Time PCR System, including a step of 94° C. hot start for 5 min followed by 35 cycles of 94ºC for 45 s, 57ºC for 45 s and further extension at 72ºC for 10 min. Relative levels of expression were determined by normalization to GAPDH, using the ΔΔCt method. Primer sequences are shown in Table 1.

TABLE 1
Primer sequences.

| | | SEQ | | SEQ |
|---|---|---|---|---|
| | | ID | | ID |
| GENE | FORWARD PRIMER SEQUENCE | NO# | REVERSE PRIMER SEQUENCE | NO# |
| GAPDH | 5′-GAGTCAACGGATTTGGTCGT-3′ | 1 | 5′-TTGATTTTGGAGGGATCTCG-3′ | 2 |
| ALPL | 5′-CGCACGGAACTCCTGACC-3′ | 3 | 5′-GCCACCACCACCATCTCG-3′ | 4 |
| HEY1 | 5′-ATGGTGTGCGAGTGGTCAAT-3′ | 5 | 5′-ACCACAGTTCCATGCACCAA-3′ | 6 |
| BMP2 | 5′-GAGGTCCTGAGCGAGTTCGA-3′ | 7 | 5′-TCTCTGTTTCAGGCCGAACA-3′ | 8 |
| BMP4 | 5′-GCCCGCAGCCTAGCAA-3′ | 9 | 5′-CGGTAAAAGATCCCGCATGTAG-3′ | 10 |
| NOG | 5′-CTGGTGGACCTCATCGAACA-3′ | 11 | 5′-CGTCTCGTTCAGATCCTTTTCCT-3′ | 12 |
| LEF1 | 5′-GCAGCTATCAACCAGATTC-3′ | 13 | 5′-GATGTAGGCAGCTGTCATTC-3′ | 14 |
| VCAN | 5′-CCAGCAAGCACAAAATTTCA-3′ | 15 | 5′-TGCACTGGATCTGTTTCTTCA-3′ | 16 |

### In Vivo Hair Induction Assays

### Statistical Analysis

Data were presented in means with standard deviation or means with a standard error of means. One-way ANOVA with Bonferroni's post-hoc tests was performed to compare the differences of gene expression level across different groups, where p<0.05 was considered statistically significant. GraphPad Prism 8.0 (GraphPad Software, San Diego, CA, USA) was used to conduct the statistical analysis.

### Results

### Dermal Papilla Cell Culture and Characterization

The hair inductive property of dermal papilla cells had been demonstrated in a few hair-reconstitution studies, however, the capacity of DPCs to induce hair regeneration was found to gradually be lost in conventional monolayer culture (Ohyama et al., 2012). To monitor the phenotype of DPCs in monolayer culture, a serial of molecular signature markers related to hair inductivity including alkaline phosphatase, versican, fibronectin, et cetera, were used to characterize the cells in 2D culture, visualized via phase-contrast image of the DPC in 2D culture showing elongated morphology (data not shown). When culturing in 2D, DPCs displayed a flattened spread-out, polygonal cellular morphology, and tended to form multilayer aggregates as shown that there were several overlaps. The aggregative morphology of dermal papilla cells was still seen in passage 5. Despite a gradual attenuation in the expression level of some signature markers, such as fibronectin and BMP2, all of the five markers were still present in passage 5. To maintain the hair inductivity of DPCs, cells no later than passage 5 were used for the experiment.

### Preparation of Collagen-DPC Microspheres

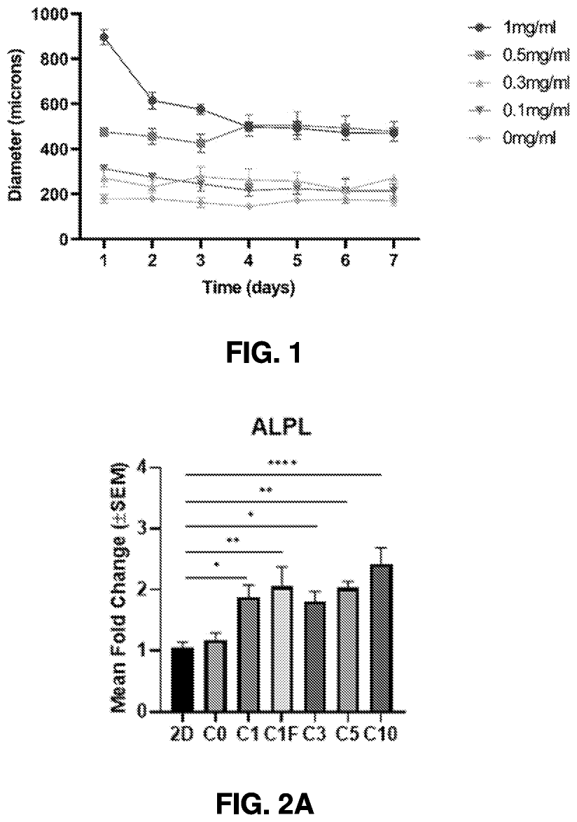

To closely resemble the native DP, the number of cells per microsphere was set to be 1250 cells/microsphere, which is similar to the number of DPCs in the human scalp hair follicle (˜1280 cells/DP) (Elliott et al., 1999). Different collagen concentrations ranging from 0.1 mg/ml to 1 mg/ml were used to prepare the collagen-DPC microspheres. In general, the more collagen incorporated into the cell-matrix mixture, the larger the size of the microspheres, but differences were shrinking as the culture prolonged because collagen-DPC microspheres gradually contracted in culture, visualized by the gross appearance of DPC microspheres at different collagen concentration. Images were captured after 2 days of culture (data not shown). Microspheres with higher collagen concentration showed significant contraction before reaching constant size while microspheres with low collagen concentration (below 0.3 mg/ml) remained a stable and constant size during the culture (FIG. 1). Dermal papilla cells alone without collagen can also self-assemble into a microsphere. Although some cell aggregates showed similar size to that of the collagen-DPC microsphere in other groups, there were a lot more tiny cell aggregates floating in the medium, which implied the instability of cell aggregates and inefficiency of applying cell aggregates for DP reconstitution. It is noted that the size of collagen-DPC microspheres with low collagen concentration (below 0.3 mg/ml) was comparable to the native human hair follicle, where the diameter of the inferior portion is about 0.25 mm (Jimenez et al., 2011).

### Restoration of DPC Molecular Signatures and Hair Follicle Inductivity

The hair follicle dermal papilla (DP) is composed of tightly packed dermal papilla cells (DPCs) and a unique extracellular matrix. In order to determine how well the collagen-DPC microsphere recapitulates the native DP, immunostaining of DPC for molecular signatures, as well as some signaling molecules, that correlate with hair inductivity was performed. Alkaline phosphatase is frequently used as an indicator of trichogenicity, as its activity is highly correlated with the hair cycle, reaching highest in early-anagen, dropping to approximately half after mid-anagen, and declining or absent in alopecia cases (Handjiski et al., 1994). Versican, a unique extracellular matrix of DP niche, is specifically present in human hair follicles during anagen and is absent in the miniaturized hair follicles of androgenetic alopecia (Soma et al., 2005). Similarly, fibronectin is stained strong during anagen while weak during catagen and telogen. (Messenger et al., 1991) The activity of Wnt and BMP signaling is also found relevant to the trichogenicity of DPCs (Ohyama et al., 2010).

For collagen-DPC microsphere with relatively high collagen concentration (above 0.5 mg/ml), dermal papilla cells were observed either concentrating in the center or distributing along the periphery of the microsphere, indicating their strong tendency to establish close cell-cell interaction (data not shown). Observations were made via immunofluorescence staining of DP phenotype markers (fibronectin and alkaline phosphatase) in DPC-microspheres (0.5 mg COL). It was observed that both ALP and fibronectin, two of the markers indicating hair inductivity, were expressed strongly only in cells that were densely packed but not in cells that were sparsely distributed within the collagen matrix. This result indicates that a rich collagen meshwork is not preferred as the matrix itself may impede the formation of close cell-cell interactions, which is crucial for the phenotype maintenance of dermal papilla cells.

In contrast with the collagen rich DPC microspheres, DPC microspheres with minimal collagen and/or fibronectin displayed a more biomimetic structure that closely resembles the native hair follicle (data not shown). Observations were made via immunofluorescence staining of DP phenotype markers (fibronectin, alkaline phosphatase, and versican) and signaling molecules (bone morphogenetic protein 2, β-catenin) in DPC-microspheres (0.1 mg COL+0.05 mg FN). In the low collagen groups, cells surrounded the collagen matrix in their self-assembled microsphere but were not entrapped in the matrix. Instead of encapsulation, here collagen acted more like the glue that facilitated the aggregation of cells and formation of microspheres. Dermal papilla cells were observed more evenly distributed within the microsphere, where ALP, BMP2, and β-catenin were stained strong throughout the microsphere. The expression of Wnt signaling-related molecule β-catenin was observed intensified in microspheres in comparison to 2D, indicating that the 3D culture environment leads to activation of the Wnt signaling pathway, which plays an important role in hair follicle morphogenesis and regeneration. Fibronectin, unlike those sparse, patchy expressions in monolayer culture, was highly expressed and displayed beautiful network structures. The expression of versican showed a diffusive pattern but not concentrating in the nucleus, which is similar to the pattern shown in early-cultured DPCs. Taking all these into consideration, it was realized that DPC microspheres with minimal matrix components embody a better microenvironment to preserve the hair inductivity of DPCs.

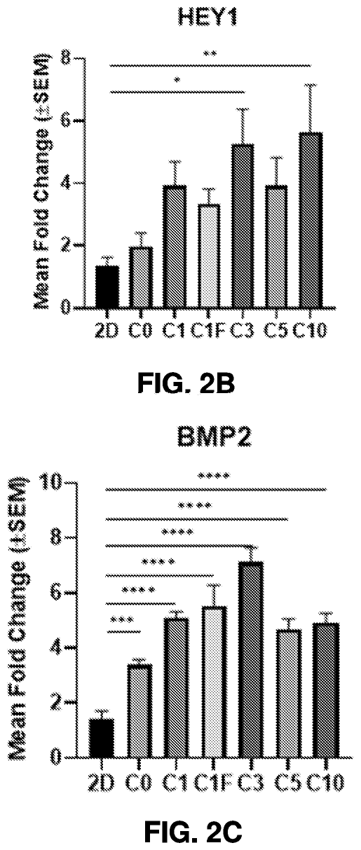

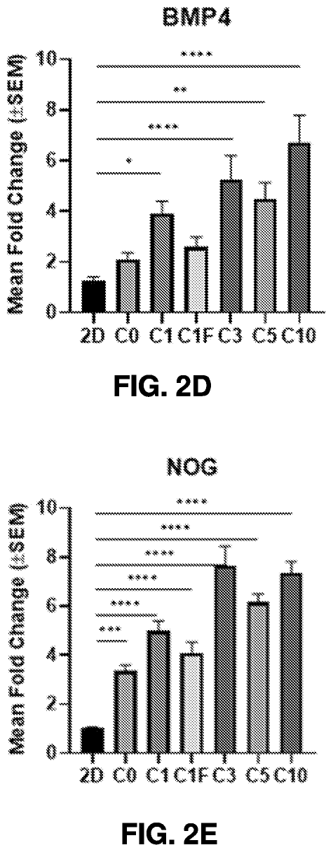

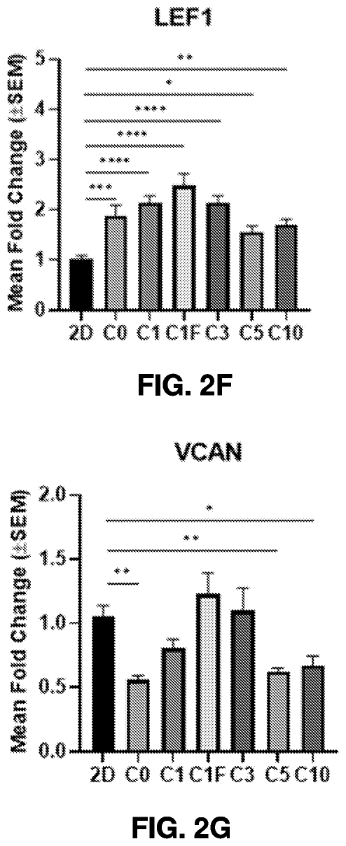

To further demonstrate that collagen-DPC microspheres restore DPC molecular signatures and hair follicle inductivity, the mRNA levels of a number of DP signature genes (ALPL, HEY1), BMP-related genes (BMP2, BMP4, and NOG), and Wnt-related genes (LEF1, VCAN) were quantified to compare the DPC microspheres and the monolayer DPCs (FIGS. 2A-2G) (Ohyama et al., 2012; Yang et al., 2012).

Compared to 2D-cultured DPCs, 3D-cultured DPCs both in the form of cell aggregates and collagen-DPC microspheres showed an upregulation in several DP signature genes, which was consistent with our immunostaining results. The expression of the ALPL gene was significantly upregulated in all collagen-DPC microsphere groups in comparison to 2D but was not significant in the cell aggregate group. Microspheres with collagen concentrations of 0.3 mg/ml and 1 mg/ml showed a significant upregulation of the HEY1 gene compared to 2D cells, and the overall expression of HEY1 in all collagen groups was higher than in the cell aggregate group. When it comes to BMP signaling-related genes, all groups in 3D culture showed a significant upregulation of BMP2 and NOG genes compared to 2D, while upregulation of BMP4 gene was observed in most collagen-DPC microsphere groups but not in cell aggregate. BMP signaling has been suggested to be important for maintaining dermal papilla cell fate and their hair-follicle-inductive capabilities and is essential for the control of cell lineage commitment and cell differentiation of epithelial progenitor cells during hair follicle development (Botchkarev & Sharov, 2004; Kobielak et al., 2003; Rendl et al., 2008). The upregulations of BMP signaling-related genes of our 3D culture collagen-DPC microspheres on the gene level indicates that our 3D culture approach is useful for preserving the DPC phenotypes and providing a niche critical for instructing epithelial progenitors to proper hair differentiation. LEF1 has been recognized as an essential regulatory protein in the Wnt signaling pathway and found to contribute to the hair differentiation of hair follicle bulge stem cells (Zhang et al., 2013). The gene expression of LEF1 showed a significant upregulation in all 3D groups as compared to 2D control, indicating that Wnt signaling is activated along with 3D culture, which is consistent with the immunostaining results here. Surprisingly, the expression of VCAN gene did not show an upregulated pattern as other genes. In contrast, it even showed a drop in cell aggregate and collagen-DPC microspheres with high collagen concentrations (0.5 mg/ml and 1 mg/ml) groups. Unlike the genes that encode signaling molecules, VCAN gene controls the expression of an extracellular matrix, versican, which has been suggested to be regulated by the Wnt/β-catenin signaling pathway and plays an important role in hair follicle development (Yang et al., 2012). It was realized that, since collagen is also an important component of the extracellular matrix, and because both collagen and versican may have some similar effects on DPCs, a rich collagen matrix as in the collagen-DPC microspheres here could provide negative feedback inhibiting the expression of versican gene. This realization is consistent with the downregulation of VCAN gene being seen only in collagen-DPC microspheres with rich collagen but not in groups with minimum collagen.

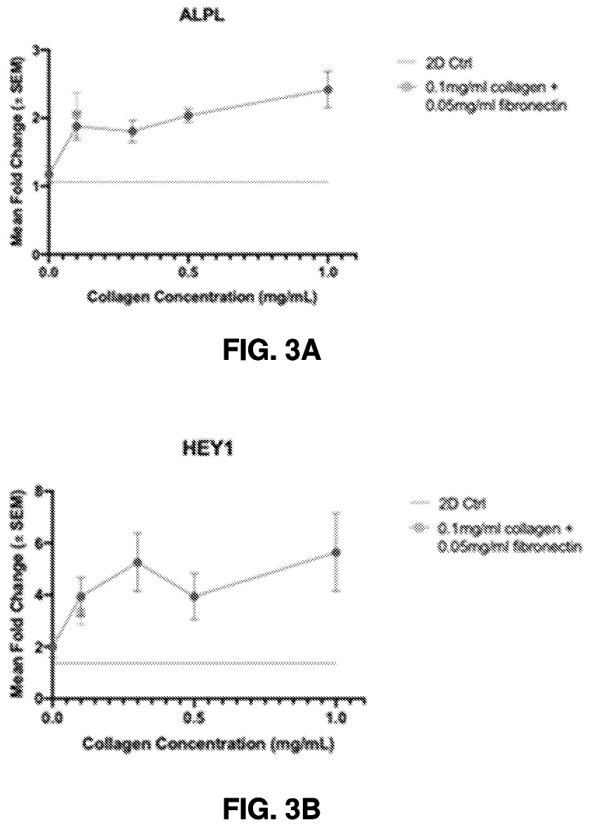

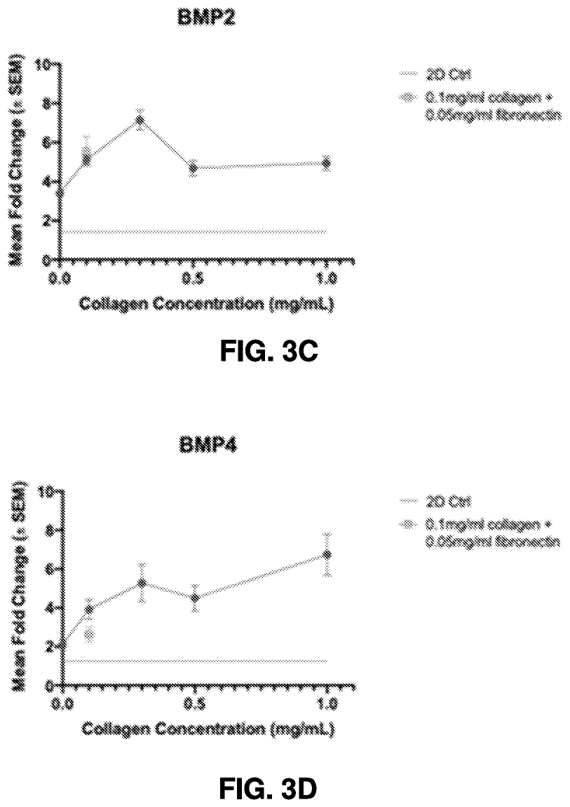

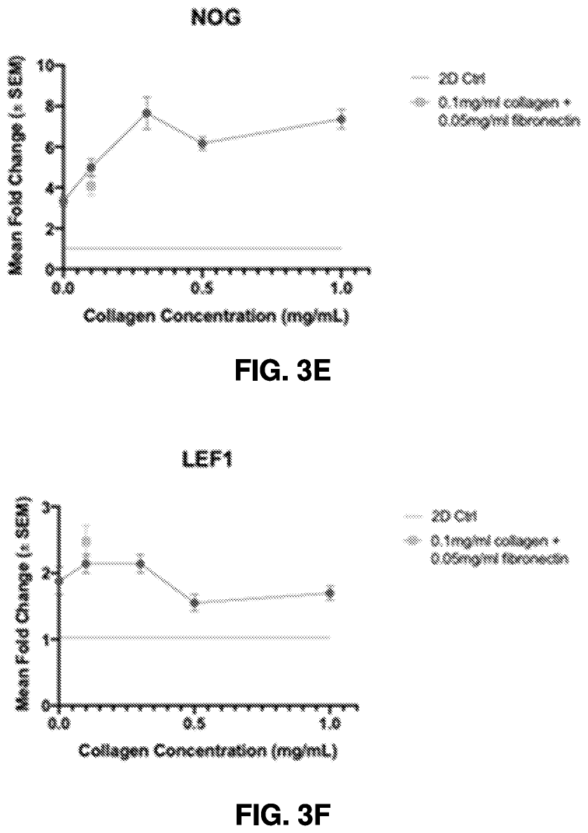

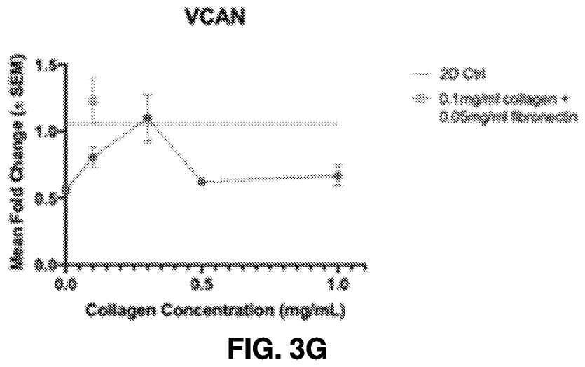

We further investigated whether the signature gene expression of collagen-DPC microspheres has some dose-dependent pattern (FIGS. 3A-3G). Interestingly, it was observed that several of the investigated genes displayed a similar increase-decrease-increase pattern, showing peaks at around 0.3 mg/ml, drops at around 0.5 mg/ml then gradually recovery at around 1 mg/ml. It was realized that a collagen dosage (i.e., amount) between 0.1 mg/ml to 0.5 mg/ml exhibits the most profound effects on DP signature gene expressions. The addition of fibronectin into the collagen-DPC microspheres seemed not to affect the gene expression much in most of the investigated genes, but it showed an enhancement in VCAN gene expression. In general, although all 3D-cultured DPCs showed elevated DP signature gene expressions compared to 2D cultures, the collagen-DPC microspheres outperform cell aggregate in terms of restoring the DP signature genes therefore the hair inductive properties.

Induction of Epithelial Cells into Hair Follicle Differentiation

The development of hair follicles requires both mesenchymal and epithelial components and their complicated mesenchymal-epithelial interactions. To establish a physiologically relevant bioengineered HF, human epidermal keratinocytes were seeded over the previously formed collagen-DPC microsphere. When co-culturing with the collagen-DPC microsphere, epidermal keratinocytes first self-assembled into several cell aggregates, and those aggregates kept merging and growing bigger, and finally attached to the collagen-DPC microsphere. After combining with the collagen-DPC microsphere, the epithelial component started growing bigger and more elongated, displaying a solid tubular structure morphologically akin to in vivo hair follicle (data not shown). The length of the protruding epithelial component could reach more than 250 μm after 3 days of co-culture and could grow longer along with the culture time.

Cross-sections of the co-cultured HF microspheres were stained with several DP markers, keratinocyte markers, and signaling molecules. DPCs after the addition of keratinocytes exhibited sustained ALP activity but the cells were more concentrated at the periphery of the microsphere where epidermal keratinocytes were attached. The epidermal keratinocytes surrounded the DPC microsphere and were concentrated in the tubular structure as indicated by the positive cytokeratin 5 staining (data not shown). The microspheres were visualized via confocal images of immunofluorescence staining of DP phenotype markers (alkaline phosphatase, fibronectin), signaling molecules (bone morphogenetic protein 2, β-catenin), proliferation markers of epithelial stem cells (cytokeratin, Integrin α6), and hair-differentiation marker (keratin 75). Culturing of the bioengineered HF microspheres for a week resulted in the differentiation of epidermal keratinocytes into hair lineages, as indicated by the positive immunostaining of hair-specific marker keratin 75. The expression of keratin 75 was the highest in the area where keratinocytes made contact with the DPCs, and the intensity gradually attenuated at the distal part of the tubular structure. Interestingly, we observed localization of fibronectin and integrin α6 at the junction area of DPC microsphere and keratinocytes aggregates, which displayed a ring-like pattern (data not shown). The Wnt signaling molecule β-catenin was detected diffusive throughout the whole HF microsphere but was not as intense as in the collagen-DPC microspheres to which keratinocytes were not co-cultured. On the contrary, the BMP signaling molecule BMP2 was strongly stained, particularly in the protruded tubular structure where keratinocytes were thought to be undergoing hair differentiation. This observation is consistent with the critical role of BMP2 in orchestrating cell commitment and cell differentiation of epithelial progenitors (Botchkarev & Sharov, 2004; Kobielak et al., 2003; Rendl et al., 2008) and is indicative that the HF microspheres faithfully exhibit characteristics of native hair follicles.

### Responses of Bioengineered Hair Follicles to Minoxidil

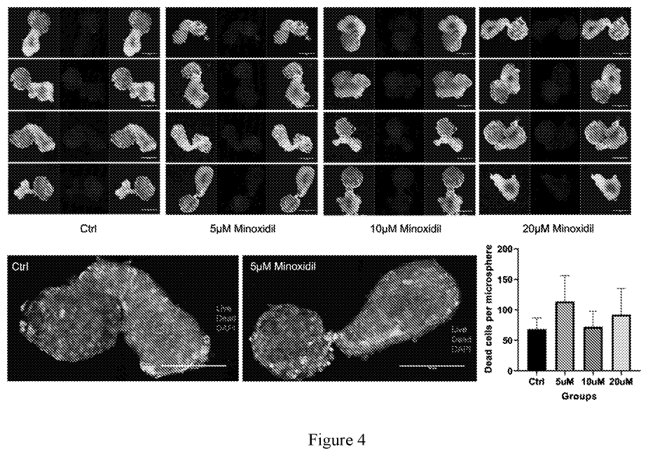

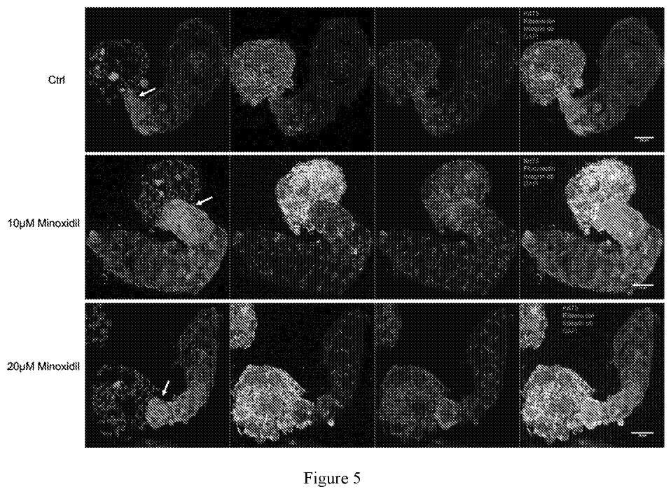

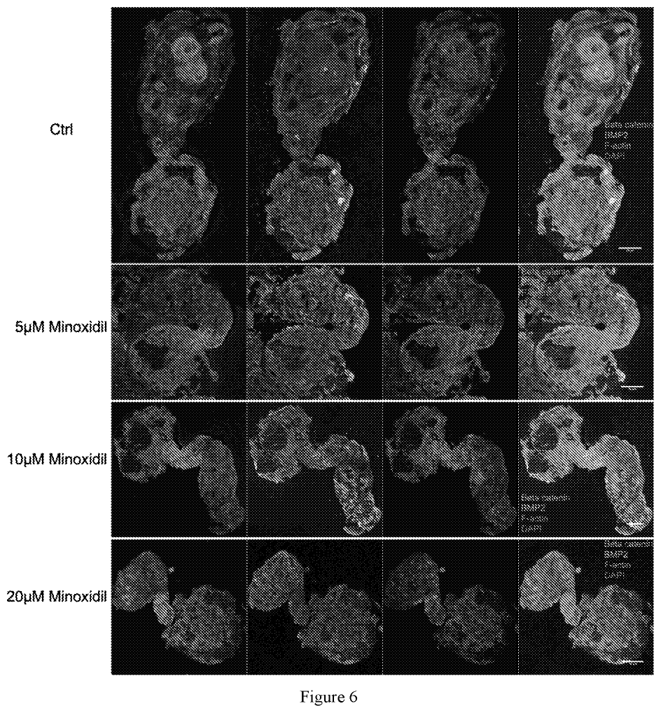

To demonstrate the feasibility of applying as in vitro drug screening model, the bioengineered hair follicle model was evaluated for cytotoxicity and marker expressions after adding well-acknowledged hair-growth stimulating drugs. Minoxidil is an FDA-approved topical drug for hair loss treatment introduced for more than 30 years. Multiple mechanisms of action of minoxidil on stimulating hair growth has been proposed, including vasodilation effects via opening of the ATP-sensitive potassium channel thus leading to an increased oxygen and nutrient supply, activation on Wnt/β-catenin signaling in DPCs thus prolonging anagen phase, stimulation on VEGF and prostaglandin E₂ synthesis by DPCs, promotion on DNA synthesis and cell proliferation of both DPCs and keratinocytes, and suppression on immunological activities in HF niche. Cell viability test using Live/Dead staining did not reveal significant changes in cell death upon minoxidil administration under the 3D configuration of our bioengineered hair follicle model. As shown from FIG. 4, cell viability test using Live/Dead staining revealed no difference in dead cell number in all microsphere groups, indicating negligible cytotoxicity upon minoxidil administration, which agreed with previous study.

The bioengineered hair follicle model also enabled examinations on the expression level of a series of hair follicle molecule signatures and signaling factors under a physiological-relevant 3D configuration, such as keratin 75 and β-catenin upon exposure to drugs interfering with the hair follicle regeneration, for example, minoxidil. Besides, VEGF secretion will also be examined as an indicator related to hair inductive property in the subsequent study.

FIG. 5 showed that microspheres applied with 10 μM or 20 μM minoxidil displayed higher expressions of hair differentiation marker Krt75 compared to the control. A profound intensification of Krt75 expression was observed at the proximal part of tubular structure (indicated by arrows) attaching to the DP component, while enhancement at the distal part was less apparent, implicating that minoxidil might facilitate hair differentiation of epithelial cells via activation of hair inductive DPCs. Minoxidil has been suggested playing a role in keratinocyte differentiation, and our study through a biomimetic in vitro HF model instead of monolayer cellular system further demonstrates the induction role of minoxidil on epithelial stem cells towards hair-lineage differentiation.

Augmented deposition of fibronectin in the collagen-DPC microsphere in groups with minoxidil application suggested that fibronectin synthesis of DPCs could be stimulated by minoxidil, and thus contributing to their enhanced hair inductive capacity.

Compared to the control, 10 μM and 20 μM minoxidil supplemented groups demonstrated an enhanced expression of integrin α6 at the junctional zone of collagen-DPC microsphere and HEKn component. Given that integrin α6 was recognized as a stem cell signature that commonly enriched in epithelial stem cell populations including hair follicle stem and progenitor cells, increase of this marker activity might indicate a higher proliferative potential and potency of HEKn with minoxidil application. Integrin α6 is also a constituent component of hemidesmosomes (HDs) that addresses importance in dermal-epidermal junction. As HDs primarily strengthen adhesion of epithelia to the basement membrane, elevated integrin α6 expression might indicate a stronger and more stable attachment of HEKn to the matrix of collagen-DPC microspheres by influence of minoxidil, which could facilitate the mesenchymal-epithelial interactions and further improve the regenerative capacity of bioengineered hair follicles.

Application of minoxidil to microspheres also resulted in a moderate augmentation of β-catenin expression near the mesenchymal-epithelial junctional region, as shown in FIG. 6, and it seemed more prominent in higher dosage groups (10 μM and 20 μM). This result conformed with a prior study reporting activation of β-catenin pathway in 2D cultured human DPCs by minoxidil and thus prolongation of anagen hair cycle. Our drug screening tests on 3D model further supported the stimulation of Wnt/β-catenin signaling by minoxidil in bioengineered hair follicles, thus potentiating hair growth and hair regeneration.

Overall, examination of marker expressions using our bioengineered follicle model reflected the positive effects of minoxidil to hair follicle growth in terms of enhancement in hair differentiation marker expression, fibronectin deposition, integrin α6 expression and Wnt/β-catenin signaling pathway. And our results agreed with previous studies documenting the stimulative roles minoxidil played on hair regeneration, suggesting that the drug screening performed on our bioengineered hair follicle model produced reliable in vitro responses at least partially resembled the clinical outcomes. And our model also offered an economical and versatile tool to study the pharmacological effects of hair growth promoters or inhibitors from different perspectives.

### In Vivo Hair Induction Assays

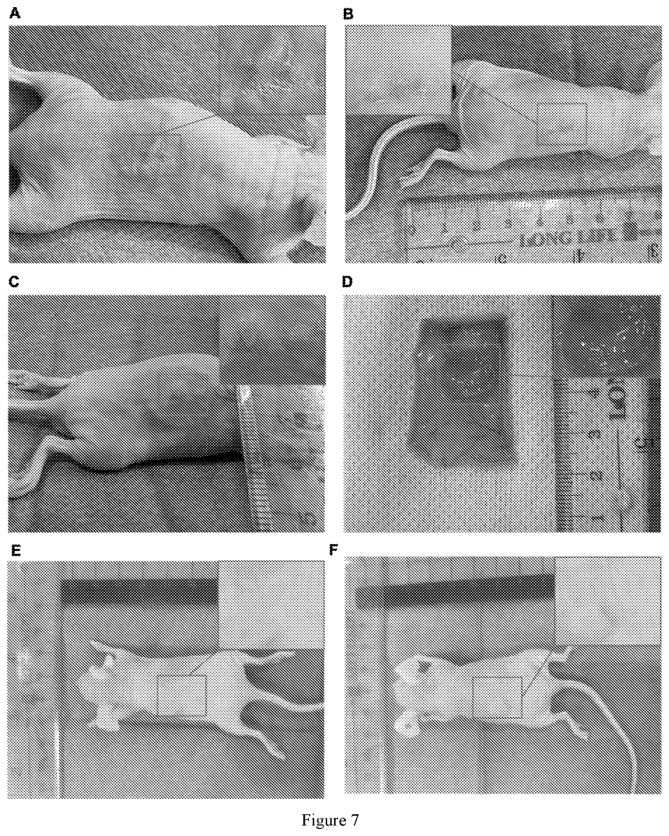

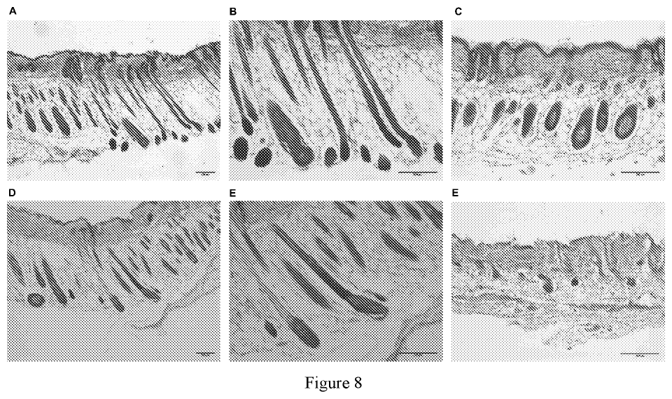

The gross appearance of nude mice dorsal skin at three weeks post-operation was shown in FIG. 7. After three weeks, new hair shafts were observed at the implantation site on mice injected with bioengineered hair follicles, but no visible hairs were observed in the injection area for the control group injected with 2D cell suspension (FIGS. 7A-7C, 7E-7F). The integrity of photo-crosslinked collagen membrane was remained after 3 weeks post-implantation (FIG. 7D). Though unpigmented and short in length, those hairs grown near or in the membrane area were noticeable on the nude mice dorsal skin that barely coated with hairs. Histological staining results further confirmed that the skin samples from microsphere group at three weeks contained plenty of hair follicles in anagen phase which were featured by an enlarged DP in oval shape, the presence of relatively wide hair matrix and enrichment of GAG shown by intense Safranin O positive staining (FIG. 8). Some hair follicles growing from deep of the subcutaneous layer were able to reach the level of sebaceous glands, indicating their maturation with accessory glands incorporated and ready to penetrate out of the epidermis. In the control group injected with DPCs and HEKn suspension at three weeks, only a limited number of hair bud-like structures were observed, which were small and enriched in GAG. To determine if the implanted DPC-HEKn microspheres constituted the newly generated hairs or induced hair growth through paracrine secretion, immunofluorescence staining using human-specific antibody will be conducted in the subsequent study.

### CONCLUSIONS

In this study, a novel method for preparing dermal papilla microspheres and bioengineered hair follicles with morphological and molecular features like native tissue is described. The collagen-DPC microspheres were formed with controllable and uniform micro-size structures similar to that of the native hair follicle, featuring elevated expression level of dermal papilla signature markers in both protein and gene levels, and enabling extensive cell-cell contacts as well as cell-matrix interactions. The bioengineered hair follicles featured a tubular structure and recapitulated hair-specific keratin expressions, demonstrating its similarities to in vivo hair follicles both structurally and ultra-structurally. The bioengineered hair follicles developed by our method demonstrated a number of advantages, including morphological and molecular relevance, minimum cell requirement, simple manipulation, and consistent, reliable production. This 3D in vitro model of hair follicles is not only useful for hair follicle research, like investigating hair follicle stem cell fate determination, but also for medical, pharmaceutical, and cosmetics applications, such as transplantation and drug screening.

### REFERENCES

Aghaei, S., Saki, N., Daneshmand, E., & Kardeh, B. (2014). Prevalence of Psychological Disorders in Patients with Alopecia Areata in Comparison with Normal Subjects. ISRN Dermatology, 2014, 1-4. doi.org/10.1155/2014/304370.
Botchkarev, V. A., & Sharov, A. A. (2004). BMP signaling in the control of skin development and hair follicle growth. Differentiation, 72(9-10), 512-526. doi.org/10.1111/j.1432-0436.2004.07209005.x.
Cartwright, T., Endean, N., & Porter, A. (2009). Illness perceptions, coping and quality of life in patients with alopecia. British Journal of Dermatology, 160(5), 1034-1039. doi.org/10.1111/j.1365-2133.2008.09014.x.
Castro, A. R., & Logarinho, E. (2020). Tissue engineering strategies for human hair follicle regeneration: How far from a hairy goal? Stem Cells Translational Medicine, 9(3), 342-350. doi.org/10.1002/sctm. 19-0301.
Driskell, R. R., Clavel, C., Rendl, M., & Watt, F. M. (2011). Hair follicle dermal papilla cells at a glance. Journal of Cell Science, 124(8), 1179-1182. doi.org/10.1242/jcs.082446.
Elliott, K., Messenger, A. G., & Stephenson, T. J. (1999). Differences in Hair Follicle Dermal Papilla Volume are Due to Extracellular Matrix Volume and Cell Number: Implications for the Control of Hair Follicle Size and Androgen Responses. Journal of Investigative Dermatology, 113(6), 873-877. doi.org/https://doi.org/10.1046/j. 1523-1747.1999.00797.x.
Gangatirkar, P., Paquet-Fifield, S., Li, A., Rossi, R., & Kaur, P. (2007). Establishment of 3D organotypic cultures using human neonatal epidermal cells. Nature Protocols, 2(1), 178-186. doi.org/10.1038/nprot.2006.448.
Gokalp, H. (2017). Psychosocial Aspects of Hair Loss. In Hair and Scalp Disorders. InTech.doi.org/10.5772/66156.
Handjiski, B. K., Eichmuller, S., Hofmann, U., Czarnetzki, B. M., & Paus, R. (1994). Alkaline phosphatase activity and localization during the murine hair cycle. British Journal of Dermatology, 131(3), 303-310. doi.org/10.1111/j.1365-2133.1994.tb08515.x.
Higgins, C. A., Chen, J. C., Cerise, J. E., Jahoda, C. A. B., & Christiano, A. M. (2013). Microenvironmental reprogramming by three-dimensional culture enables dermal papilla cells to induce de novo human hair-follicle growth. Proceedings of the National Academy of Sciences of the United States of America, 110(49), 19679-19688. doi.org/10.1073/pnas. 1309970110.
Hunt, N., & McHale, S. (2005). The psychological impact of alopecia. British Medical Journal, 331(7522), 951-953. doi.org/10.1136/bmj.331.7522.951.
Jimenez, F., Izeta, A., & Poblet, E. (2011). Morphometric Analysis of the Human Scalp Hair Follicle: Practical Implications for the Hair Transplant Surgeon and Hair Regeneration Studies. Dermatologic Surgery, 37(1), 58-64. doi.org/10.1111/j.1524-4725.2010.01809.x.
Kobielak, K., Pasolli, H. A., Alonso, L., Polak, L., & Fuchs, E. (2003). Defining BMP functions in the hair follicle by conditional ablation of BMP receptor IA. The Journal of Cell Biology, 163(3), 609-623. doi.org/10.1083/jcb.200309042.
Messenger, A. G., Elliott, K., Westgate, Gi. E., & Gibson, W. T. (1991). Distribution of Extracellular Matrix Molecules in Human Hair Follicles. Annals of the New York Academy of Sciences, 642(1), 253-262. doi.org/10.1111/j.1749-6632.1991.tb24392.x.
Ohyama, M., Kobayashi, T., Sasaki, T., Shimizu, A., & Amagai, M. (2012). Restoration of the intrinsic properties of human dermal papilla in vitro. Journal of Cell Science, 125(17), 4114-4125. doi.org/10.1242/jcs. 105700.
Ohyama, M., Zheng, Y., Paus, R., & Stenn, K. S. (2010). The mesenchymal component of hair follicle neogenesis: Background, methods and molecular characterization. Experimental Dermatology, 19(2), 89-99. doi.org/10.1111/j.1600-0625.2009.00935.x.
Premanand, A., & Reena Rajkumari, B. (2018). Androgen modulation of Wnt/β-catenin signaling in androgenetic alopecia. Archives of Dermatological Research, 310(5), 391-399. doi.org/10.1007/s00403-018-1826-8.
Reddy, S., Andl, T., Bagasra, A., Lu, M. M., Epstein, D. J., Morrisey, E. E., & Millar, S. E. (2001). Characterization of Wnt gene expression in developing and postnatal hair follicles and identification of Wnt5a as a target of Sonic hedgehog in hair follicle morphogenesis. Mechanisms of Development, 107(1-2), 69-82. doi.org/10.1016/S0925-4773(01)00452-X.
Rendl, M., Polak, L., & Fuchs, E. (2008). BMP signaling in dermal papilla cells is required for their hair follicle-inductive properties. Genes & Development, 22(4), 543-557. doi.org/10.1101/gad.1614408.
Sadick, N. S., Callender, V. D., Kircik, L. H., & Kogan, S. (2017). New insight into the pathophysiology of hair loss trigger a paradigm shift in the treatment approach. Journal of Drugs in Dermatology, 16(11), s135-$140.
Soma, T., Tajima, M., & Kishimoto, J. (2005). Hair cycle-specific expression of versican in human hair follicles. Journal of Dermatological Science, 39(3), 147-154. doi.org/10.1016/j.jdermsci.2005.03.010.
Williamson, D., Gonzalez, M., & Finlay, A. Y. (2001). The effect of hair loss in quality of life. Journal of the European Academy of Dermatology and Venereology, 15(2), 137-139. doi.org/10.1046/j.1468-3083.2001.00229.x.
Yang, Y., Li, Y., Wang, Y., Wu, J., Yang, G., Yang, T., Gao, Y., & Lu, Y. (2012). Versican gene: Regulation by the β-catenin signaling pathway plays a significant role in dermal papilla cell aggregative growth. Journal of Dermatological Science, 68(3), 157-163. doi.org/10.1016/j.jdermsci.2012.09.011.
Zhang, Y., Yu, J., Shi, C., Huang, Y., Wang, Y., Yang, T., & Yang, J. (2013). Lef1 contributes to the differentiation of bulge stem cells by nuclear translocation and cross-talk with the notch signaling pathway. International Journal of Medical Sciences, 10(6), 738-746. doi.org/10.7150/ijms.5693.
Han, J. H. et al. Effect of minoxidil on proliferation and apoptosis in dermal papilla cells of human hair follicle. Journal of Dermatological Science 34, 91-98 (2004).
Boyera, N., Galey, I. & Bernard, B. A. Biphasic effects of minoxidil on the proliferation and differentiation of normal human keratinocytes. Skin Pharmacology and Physiology 10, 206-220 (1997).
Messenger, A. G. & Rundegren, J. Minoxidil: Mechanisms of action on hair growth. British Journal of Dermatology vol. 150 186-194 (2004).
Krebsbach, P. H. & Villa-Diaz, L. G. The Role of Integrin α6 (CD49f) in Stem Cells: More than a Conserved Biomarker. Stem Cells and Development 26, 1090 (2017).
Garza, L. A. et al. Bald scalp in men with androgenetic alopecia retains hair follicle stem cells but lacks CD200-rich and CD34-positive hair follicle progenitor cells. Journal of Clinical Investigation 121, 613-622 (2011).
Briggaman, R. A. & Wheeler, C. E. The epidermal-dermal junction. J Invest Dermatol 65, 71-84 (1975).
Aleemardani, M., Trikić, M. Z., Green, N. H. & Claeyssens, F. The Importance of Mimicking Dermal-Epidermal Junction for Skin Tissue Engineering: A Review. Bioengineering 8, (2021).
Nievers, M. G., Schaapveld, R. Q. J. & Sonnenberg, A. Biology and function of hemidesmosomes. Matrix Biology 18, 5-17 (1999).
Kwack, M. H., Kang, B. M., Kim, M. K., Kim, J. C. & Sung, Y. K. Minoxidil activates β-catenin pathway in human dermal papilla cells: A possible explanation for its anagen prolongation effect. Journal of Dermatological Science 62, 154-159 (2011).
Oh, J. W. et al. A guide to studying human Hair follicle cycling in vivo. Journal of Investigative Dermatology 136, 34-44 (2016).
Couchman, J. R. Hair follicle proteoglycans. Journal of Investigative Dermatology 101, 60-64 (1993).

It is understood that the disclosed method and compositions are not limited to the particular methodology, protocols, and reagents described as these can vary. It is also to be understood that the terminology used herein is for the purpose of describing particular embodiments only and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compositions may not be explicitly disclosed, each is specifically contemplated and described herein. For example, if a bioengineered hair follicle is disclosed and discussed and a number of modifications that can be made to a number of compositions including the bioengineered hair follicle are discussed, each and every combination and permutation of bioengineered hair follicles and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms “a,” “an,” and “the” include plural reference unless the context clearly dictates otherwise. Thus, for example, reference to “a bioengineered hair follicle” includes a plurality of such bioengineered hair follicles, reference to “the bioengineered hair follicle” is a reference to one or more bioengineered hair follicles and equivalents thereof known to those skilled in the art, and so forth.

Throughout the description and claims of this specification, the word “comprise,” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e., a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents form part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives. Thus, for example, a list of different components does not indicate that the listed components are obvious one to the other, nor is it an admission of equivalence or obviousness.

Those skilled in the art will recognize or be able to ascertain using no more than routine experimentation many equivalents to the specific embodiments of the method and compositions described herein. Such equivalents are intended to be encompassed by the following claims.

1. A method of producing a bioengineered hair follicle, the method comprising
forming a microsphere comprising mesenchymal cells and extracellular matrix by dispensing a droplet of a suspension of the mesenchymal cells and extracellular matrix into a vessel, and incubating the droplet in a culture vessel, thereby forming a mesenchymal cell-matrix microsphere;
culturing the mesenchymal cell-matrix microsphere in the vessel in the presence of supplementary factors;
dispensing to the vessel a droplet of a suspension of epithelial cells in close proximity to the mesenchymal cell-matrix microsphere to form a mesenchymal microsphere-epithelial cell mixture, and culturing the mesenchymal microsphere-epithelial cell mixture; and
changing the medium in the vessel to epidermalization medium and culturing, thereby producing a bioengineered hair follicle.

2. The method of claim 1, wherein:
(a) the droplet of the suspension of the mesenchymal cells and extracellular matrix has a volume ranging from 0.5 to 10.0 μL, from 1.0 to 5.0 μL, or from 2.0 to 3.0 L; and/or
(b) the suspension of the mesenchymal cells and extracellular matrix comprises the mesenchymal cells at a density of from 1×10⁴ to 1×10⁷ cells/ml, and extracellular matrix at a concentration of from 0.01 mg/ml to 3.0 mg/ml, 0.01 mg/ml to 2.0 mg/ml, or 0.05 mg/ml to 0.5 mg/ml.

3. (canceled)

4. The method of claim 1, wherein the mesenchymal cells are human dermal papilla cells (DPCs), human mesenchymal stem cells, human fibroblasts, or a combination thereof.

5. The method of claim 1, wherein the extracellular matrix comprises collagen, fibronectin, fibrinogen, laminin, glycosaminoglycans, vitronectin, or a combination thereof.

6. (canceled)

7. The method of claim 1, wherein the culture vessel comprises a 384 well culture plate, a custom-made 88 well microwell, or a PDMS-based microwell.

8. The method of claim 1, wherein the supplementary factors comprise FGF, HGFs, Wnt, BMP, PDGF, or a combination thereof.

9. The method of claim 1, wherein the mesenchymal cell-matrix microsphere is cultured in the vessel in the presence of supplementary factors at from 25° C. to 39ºC, optionally 37° C. in a humidified atmosphere with from 3.5% to 6% CO₂ for from 1 to 100 hours, optionally from 12 hours to 30 hours.

10. The method of claim 1, wherein the droplet of the suspension of the epithelial cells has a volume ranging from 0.5 to 10.0 μL, 1.0 to 5.0 μL, or from 2.0 to 3.0 μL, and wherein the suspension contains the epithelial cells at a density of from 1×10⁴ to 1×10⁷ cells/ml, or optionally 1×10⁵ to 1×10⁶ cells/ml.

11. The method of claim 1, wherein the mesenchymal microsphere-epithelial cell mixture is cultured at from 35° C. to 39° C. in a humidified atmosphere with from 3.5% to 6% CO₂ for from 1 hours to 100 hours, 5 hours to 50 hours, or optionally 18 hours to 30 hours.

12. The method of claim 1, wherein the epithelial cells are human epidermal keratinocytes, human hair follicle keratinocytes, human epidermal progenitor or stem cells, human iPSC derived epithelial cells, or a combination thereof.

13. The method of claim 1, wherein the ratio of mesenchymal cells to epithelial cells is from 0.1:1 to 10:1, or optionally 1:1.

14. The method of claim 1, wherein the droplets of mesenchymal cells and extracellular matrix are incubated overnight, wherein the mesenchymal cell-matrix microsphere is cultured overnight, and wherein the mesenchymal cell microsphere-epithelial cell mixture is cultured overnight.

15. The method of claim 1, wherein the mesenchymal cell microsphere-epithelial cell mixture is cultured in epidermalization medium for 8 days.

16. The method of claim 1, wherein;
(a) the droplet of mesenchymal cells and matrix contains about 500 to about 10000 cells, about 1000 to about 5000 cells, or about 1000 to about 3000 cells, or optionally about 1250 mesenchymal cells; and/or
(b) the mesenchymal cell microsphere-epithelial cell mixture contains at least one or more mesenchymal cell-matrix microsphere and about 500 to about 10000, about 1000 to about 5000, about 1000 to about 3000, or about 1250 epithelial cells.

17. (canceled)

18. The method of claim 1, wherein the mesenchymal cell-matrix microsphere has one or more features indicative of its hair inductivity, wherein optionally the one or more features indicative of the hair inductivity of the mesenchymal cell-matrix microsphere comprises expression of alkaline phosphate, expression of versican, expression of fibronectin, activation of the Wnt signaling pathway, activation of the BMP signaling pathway, or a combination thereof.

19. The method of claim 1, wherein the bioengineered hair follicle has one or more features indicative of hair inductivity, wherein optionally the one or more features indicative of hair inductivity of the bioengineered hair follicle comprises expression of alkaline phosphate, expression of fibronectin, or a combination thereof.

20. The method of claim 1, wherein the bioengineered hair follicle has one or more features indicative of proliferation of epithelial cells, wherein optionally the one or more features indicative of proliferation of epithelial cells comprises expression of cytokeratin, expression of Integrin α6, or a combination thereof.

21. The method of claim 1, wherein the bioengineered hair follicle has one or more features indicative of hair differentiation, wherein optionally the one or more features indicative of hair differentiation comprises expression of keratin 75.

22. The method of claim 1, wherein the cells in the bioengineered hair follicle have both cell-cell contacts and cell-extracellular matrix contacts.

23. The method of claim 1, wherein the mesenchymal cell-matrix microsphere comprises a spherical structure morphologically similar to native dermal papilla structure, wherein optionally the spherical structure has a diameter ranging from 50 to 2000 μm.

24. (canceled)

25. The method of claim 1, wherein the bioengineered hair follicle comprises a tubular structure morphologically similar to native hair follicles.

26. The method of claim 1, wherein the mesenchymal cell-matrix microsphere is cultured in the absence of any other mesenchymal cell-matrix microsphere in the same vessel, or in a single well in a multiwell plate.

27. A bioengineered hair follicle produced by the method of claim 1.

28. A method of using the bioengineered hair follicle of claim 27, the method comprising
contacting the bioengineered hair follicle with a test compound, measuring a feature of the bioengineered hair follicle, comparing the measured feature to the same feature measured in a control bioengineered hair follicle that was not contacted with the test compound, wherein a difference in the measured features indicates that the test compound affects the measured feature of the bioengineered hair follicle,
wherein optionally the measured feature is hair follicle growth, wherein a difference in the measure hair follicle growth indicates that the test compound affects hair follicle growth.

29. (canceled)

30. A method of using the bioengineered hair follicle of claim 27 for the prophylactic or therapeutic treatment of a state of reduced pilosity or for the treatment of alopecia.

31. (canceled)
